(19) United States
(12) Patent Application Publication
(10) Pub. No.: US 2008/0249123 A1
(43) Pub. Date: Oct. 9, 2008

(54) WORTMANNIN-RAPAMYCIN CONJUGATE AND USES THEREOF

(75) Inventors: JIANXIN GU, River Edge, NJ (US); Ker Yu, Pine Brook, NJ (US); Judy Lucas, Nanuet, NY (US); Mark Ruppen, Garnerville, NY (US); Arie Zask, New York, NY (US); Semiramis Ayral-Kaloustian, Tarrytown, NY (US)

Correspondence Address: HOWSON AND HOWSON/WYETH;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 12/061,908

(22) Filed: Apr. 3, 2008

Related U.S. Application Data
(60) Provisional application No. 60/921,907, filed on Apr. 5, 2007.

(51) Int. Cl. A61K 31/436; C07D 267/00; A61P 35/04

(52) U.S. Cl. 514/291; 540/468

(57) ABSTRACT

A rapamycin—wortmannin conjugate is described, in which the conjugate is formed by linking the rapamycin and wortmannin together in such a manner that the rapamycin and the wortmannin are separated following administration to a subject. Use of such a conjugate in an antineoplastic regimen is described.

### CROSS-REFERENCE TO RELATED APPLICATIONS

This application claims the benefit of the priority of U.S. Provisional Patent Application No. 60/921,907, filed Apr. 5, 2007.

### BACKGROUND OF THE INVENTION

The present invention relates to rapamycin-wortmannin conjugates having anti-tumor activity.

Wortmannins and Rapamycins are two classes of highly potent and specific inhibitors of phosphatidylinositol-3(OH)-kinase (PI3K) and mTOR, respectively. PI3K is a heterodimeric enzyme comprised of the p85 regulatory and p110 catalytic subunits. In response to growth factor receptor stimulation, PI3K catalyzes the production of the lipid second messenger phosphatidylinositol-3,4,5-triphosphate (PIP3) at the cell membrane. PIP3 in turn contributes to the activation of a wide range of downstream cellular substrates. The most critical signaling mediators downstream of PI3K include the serine/threonine kinase AKT and the mammalian target of rapamycin (mTOR). AKT confers a dominant survival signal and promotes proliferation via direct phosphorylation of multiple cell death/apoptosis proteins and cell cycle factors. mTOR is a central regulator of cell growth via controlling cellular protein translation. Thus, the PI3K/AKT/TOR pathway is critical for cell proliferation, growth, survival and angiogenesis.

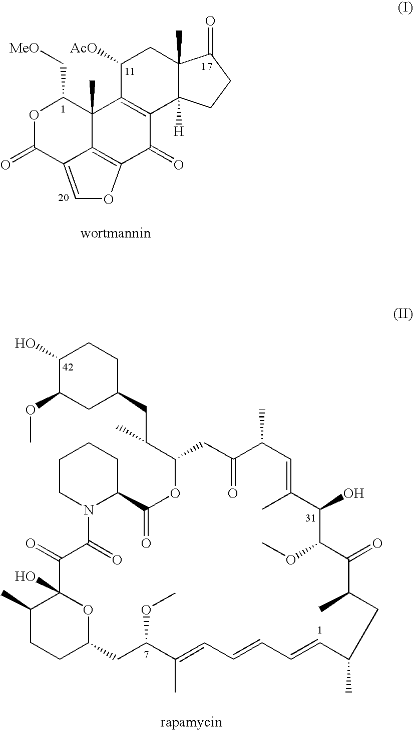

Wortmannin (formula (I)) is an irreversible inhibitor at nanomolar concentration of PI3K that binds to a lysine in the ATP binding pocket of PI3K via opening of the electrophilic furan ring at its C-20 position and has been reported to have antitumor activity against tumor xenografts in animals. [Schultz, R. M., et al, (1995) “In vitro and in vivo antitumor activity of the phosphatidylinositol-3-kinase inhibitor, wortmannin” Anticancer Res., 15, 1135-1140]. The syntheses and SAR studies of wortmannin derivatives in efforts to search for analogs with improved properties over wortmannin have been described previously. For example, a 17β-Hydroxywortmannin prepared from the reduction of wortmannin with diborane, showed a 10-fold increase in activity relative to wortmannin and pushed the PI3K IC₅₀ into the subnanomolar range, with an IC₅₀ of 0.50 nM. However, anti-tumor activity of 17β-Hydroxywortmannin in the C3H mammary model showed no inhibition at a dose of 0.5 mg/kg and toxicity at a dose of 1.0 mg/kg. These findings led the authors to conclude that nucleophilic addition to the electrophilic C-20 position of wortmannin and related analogs is required for inhibitor potency and antitumor activity, but that this mechanism appears linked to the observed toxicity. [Norman, Bryan H., et al. (1996) “Studies on the Mechanism of Phosphatidylinositol 3-Kinase Inhibition by Wortmannin and Related Analogs,” J. Med. Chem., 39, 1106-1111, 1109-1110].

Recently, it was found that pegylated 17β-hydroxywortmannin (PEG-17-HWT conjugate) demonstrated an increased tolerability as compared to 17β-hydroxywortmannin in vivo [Yu K, et al., (2005), “PWT-458, A Novel Pegylated-17-Hydroxywortmannin, Inhibits Phosphatidylinositol 3-Kinase Signaling and Suppresses Growth of Solid Tumors” Cancer Biol Ther. 4(5)].

Acetylation of 17β-hydroxywortmannin at its C-17 hydroxyl site showed a dramatic loss in activity leading the authors to conclude, “the active site cannot accommodate liphophilicity or steric bulk at C-17.” [Creemer, L. C., et al. (1996) “Synthesis and in Vitro Evaluation of New Wortmannin Esters: Potent Inhibitors of Phosphatidylinositol 3-Kinase,” J. Med. Chem., 39, 5021-5024, 5022]. This conclusion is consistent with the X-ray crystallographic structure of PI3K bound to wortmannin subsequently elucidated [Walker, Edward H., et. al. (2000) “Structural Determinants of Phosphoinositide 3-Kinase Inhibition by Wortmannin, LY294002, Quercetin, Myricetin, and Staurosporine,” Molecular Cell, 6(4), 909-919].

Other wortmannin derivatives are C-20 ring-opened compounds. By reacting wortmannin with nucleophiles at the C-20 position, the furan ring is opened. Such ring-opened compounds demonstrate a range of biological activities with improved toxicity and biological stability [Wipf, Peter, et al. (2004) “Synthesis and biological evaluation of synthetic viridins derived from C(20)-heteroalkylation of the steroidal PI-3-kinase inhibitor wortmannin,” Org. Biomol. Chem., 2, 1911-1920]. See also US Published Patent Application No. US2003/0109572 to Powis.

Rapamycin (formula (II)) is a potent mTOR inhibitor and has been reported to inhibit tumor growth [Eng, C. P., et al, (1984) “Activity of rapamycin against transplanted tumors” J. Antibiot., 37, 1231-1237]. Preclinical studies of rapamycin determined potency against many solid tumor types including breast, colon, prostate and renal cell carcinomas with typical IC₅₀<50 nM.

What are needed are alternative therapies for treatment of neoplasms.

### SUMMARY OF THE INVENTION

The invention provides a rapamycin—wortmannin conjugate. In one embodiment, the conjugate is characterized by having the formula:

Rap-L-Wort,

or a pharmaceutically acceptable salt or hydrate thereof, wherein Rap is a rapamycin; Wort is a wortmannin, and L is a linker which is bound to the rapamycin and the wortmannin. This conjugate has shown enhanced antineoplastic activity and reduced toxicity as compared to the delivery of rapamycin and a wortmannin as separate compounds.

In another aspect, a composition is provided and contains the Rap-L-Wort conjugate and a pharmaceutically acceptable carrier.

In still another aspect, the use of a rapamycin—wortmannin conjugate described herein for the preparation of a medicament useful in antineoplastic therapy is provided.

Still other aspects and advantages of the invention will be readily apparent to one of skill in the art.

### BRIEF DESCRIPTION OF THE FIGURES

FIG. 1 shows routes for the in vitro cleavage of 42,17′-linked-wortmannin-suberate-rapamycin conjugate, N,N,N′-trimethyl 1,3-propanediamine adduct in plasma. This metabolic cleavage pathway was observed by incubating the conjugate with nude mouse blood as described in Example 15.

FIG. 2 shows the sustained efficacy of 42,17-linked wortmannin-adipate-rapamycin conjugate, N,N,N′-trimethyl 1,3-propanediamine adduct against U87MG glioma xenograph in multi-cycle treatment.

FIG. 3 shows the efficacy of 42,17′-linked wortmannin-suberate-rapamycin conjugate, N,N,N′-trimethyl 1,3-propanediamine adduct; rapamycin; 17-hydroxywortmannin N,N,N′-trimethyl 1,3-propanediamine adduct; and a physical combination of rapamycin and 17-hydroxywortmannin N,N,N′-trimethyl 1,3-propanediamine adduct against U87MG glioma xenograph when dosed 1× weekly for 2 rounds.

FIG. 4 shows the efficacy of 42,17′-linked wortmannin-suberate-rapamycin conjugate, N,N,N′-trimethyl 1,3-propanediamine adduct, and rapamycin, against U87MG Glioma xenograph after a single dose at three dosing concentrations.

FIG. 5 shows the efficacy of 42,17′-linked wortmannin-suberate-rapamycin conjugate, N,N,N′-trimethyl 1,3-propanediamine adduct; rapamycin; 17-hydroxywortmannin N,N,N′-trimethyl 1,3-propanediamine adduct, and a physical combination of rapamycin and 17-hydroxywortmannin N,N,N′-trimethyl 1,3-propanediamine adduct against HT29 colon tumor xenograph when dosed 1× weekly for 4 rounds.

FIG. 6 shows the efficacy of 42,17′-linked wortmannin-suberate-rapamycin conjugate, N,N,N′-trimethyl 1,3-propanediamine adduct; Intron® A reagent; and a combination of 42,17′-linked wortmannin-suberate-rapamycin conjugate, N,N,N′-trimethyl 1,3-propanediamine adduct, and Intron® A reagent against A498 renal cell carcinoma xenograph.

FIG. 7 shows the efficacy of 42,17′-linked wortmannin-suberate-rapamycin conjugate, N,N,N′-trimethyl 1,3-propanediamine adduct; the Avastin® drug, and a combination of 42,17′-inked wortmannin-suberate-rapamycin conjugate, N,N,N′-trimethyl 1,3-propanediamine adduct and the Avastin® drug against A498 renal cell carcinoma xenograph. On day 23, the Vehicle group was redosed with a combination 42,17′-linked wortmannin-suberate-rapamycin conjugate, N,N,N′-trimethyl 1,3-propanediamine adduct and the Avastin® drug. On day 43, the 42,17-linked wortmannin-suberate-rapamycin conjugate, N,N,N′-trimethyl 1,3-propanediamine adduct group and the Avastin® drug group were redosed with a combination 42,17-linked wortmannin-suberate-rapamycin conjugate, N,N,N′-trimethyl 1,3-propanediamine adduct and the Avastin® drug.

### DETAILED DESCRIPTION OF THE INVENTION

The invention provides a rapamycin—wortmannin conjugate having the formula:

Rap-L-Wort

or a pharmaceutically acceptable salt or hydrate thereof, wherein Rap is a rapamycin; Wort is a wortmannin; and L is a linker which is bound to the rapamycin and the wortmannin. Further provided are compositions containing this conjugate and methods of using the same for preparation of medicaments useful as antineoplastic agents.

The Rap-L-Wort conjugates are anticipated to provide distinct advantages over either single agent or the physical (non-linked) combination of the two. Without wishing to be bound by theory, it is believed that in certain embodiments, the covalent linking of a wortmannin to a rapamycin will improve the solubility over the rapamycin alone. Improved solubility has important implications in clinical development and formulation. Further, as cancer therapies will likely consist of a cocktail of various combinations of compositions and standard chemotherapies, the simplicity of using a single, water-soluble dual inhibitor, rather than two separate inhibitors that require two different clinical formulations, is advantageous from the perspective of formulating the compounds. Moreover, the covalently linked Rap-L-Wort compounds are anticipated to outperform either single agent and also outperform physical combination of the two. For example, in preliminary experiments, Rap-L-Wort compounds have shown improved antineoplastic efficacy and improved tolerability over the physical combination of the two agents.

As defined herein, the term “a rapamycin” defines a class of immunosuppressive compounds which contain the rapamycin nucleus provided above. In one embodiment, the rapamycin nucleus in a conjugate of the invention is of formula (IIa):

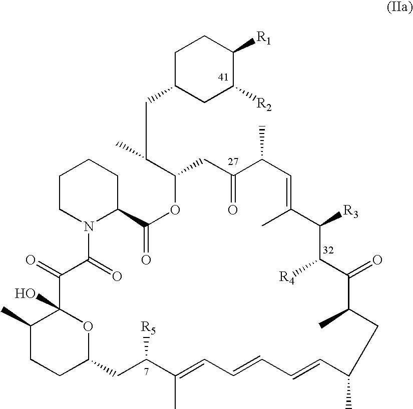

wherein, R¹ is selected from among OH, an ester, an ether, an amide, a carbonate, a carbamate, phosphate, and a tetrazole; R² is selected from among OH, an ester, and an ether; R³ is selected from among OH, an ester, an amide, a carbonate, a carbamate and an ether; R⁴ is selected from among H, OH, an ester, and an ether; R⁵ is selected from among OH, an ester, and an ether. With respect to this core, rapamycin is characterized by R¹ is OH; R² is OMe; R³ is OH; R⁴ is OMe; and R⁵ is OMe. In another embodiment, R² is selected from among OH, an ester, an ether, and a point of attachment to L.

As defined above, the term “a rapamycin” includes rapamycin and esters, ethers, amides, carbonates, carbamates, sulfonates, oximes, hydrazones, and hydroxyamines of rapamycin as well as rapamycins in which functional groups on the nucleus have been modified, for example through reduction or oxidation, replacement with a nucleophile such as tetrazole, a metabolite of rapamycin such as various desmethylrapamycin derivatives or a ring opened rapamycin (such as secorapamycin, described in U.S. Pat. No. 5,252,579). The term rapamycin also includes pharmaceutically acceptable salts of rapamycins, which are capable of forming such salts, either by virtue of containing an acidic or basic moiety.

In one embodiment, the rapamycin core defined above excludes 41-desmethoxyrapamycin.

Unless otherwise specified, an “amide” is —CONH—, where the carbon atom is generally bound to a hydrocarbon radical. Where the amide is a substituent of any of R¹-R⁵ of the rapamycin core, the N forms the point of attachment to the rapamycin core.

A “carbonate” contains a —OC(O)O— group. Where the carbonate is a substituent of any of R¹-R⁵ of the rapamycin core, one oxygen atom is generally bound to a hydrocarbon radical, and the other oxygen atom forms the point of attachment to the rapamycin core.

A “carbamate” contains a —NH(CO)O— group, where either nitrogen or oxygen is generally bound to a hydrocarbon radical. Where the carbamate is a substituent of any of R¹-R⁵ of the rapamycin core, either O or N forms the point of attachment to the rapamycin core.

A “sulfonate” contains a —S(O)₂O— group, where the S atom is generally bound to a hydrocarbon radical. Where the sulfonate is a substituent of any of the R¹-R⁵ of the rapamycin core, the O forms the point of attachment to the rapamycin core.

A “phosphate” contains a —OP(O)(OR)₂— group, where R is either alkyl, aryl, alkenyl, where the phosphate is a substituent of any of the R¹-R⁵ of the rapamycin core, the O forms the point of attachment to the rapamycin core.

An “ether” has the structure —O—, where one group on the oxygen is generally a hydrocarbon radical. Where the ether is a substituent of any of the R¹-R⁵ of the rapamycin core, the O forms the point of attachment to the rapamycin core.

An “ester” has the structure —C(O)O—, where the carbon atom is generally bound to a hydrocarbon radical. Where the ester is a substituent of any of the R¹-R⁵ of the rapamycin core, the O forms the point of attachment to the rapamycin core. One example of such an ester is where the rapamycin is CCI-779 and R¹ is an ester with 3-hydroxy-2-(hydroxymethyl)-2-methylpropionic acid.

As used herein, pharmaceutically acceptable salts include, but are not limited to, hydrochloric, hydrobromic, hydroiodic, hydrofluoric, sulfuric, citric, maleic, acetic, lactic, nicotinic, succinic, oxalic, phosphoric, malonic, salicylic, phenylacetic, stearic, pyridine, ammonium, piperazine, diethylamine, nicotinamide, formic, urea, sodium, potassium, calcium, magnesium, zinc, lithium, cinnamic, methylamino, methanesulfonic, picric, tartaric, triethylamino, dimethylamino, and tris(hydroxymethyl)aminomethane. Additional pharmaceutically acceptable salts are known to those skilled in the art.

With respect to this core (IIa), rapamycin is characterized by R¹ is OH; R² is OMe; R³ is OH; R⁴ is OMe; and R⁵is OMe.

The terms “O-desmethylrapamycin”, and “desmethylrapamycin” are used interchangeably throughout the literature and the present specification, unless otherwise specified. These terms refer to the class of immunosuppressive compounds which contain the basic rapamycin nucleus shown, but lacking one or more methyl groups. In one embodiment, the rapamycin nucleus is missing a methyl group from positions 7, 32, or 41, or combinations thereof. Production of desmethylrapamycins has been described. See, e.g., 41-desmethylrapamycin [International Patent Publication Nos. WO 2006/095185 and WO 2004/007709]. The synthesis of other desmethylrapamycins may be genetically engineered so that methyl groups are missing from other positions in the rapamycin nucleus. See, e.g., 3-desmethylrapamycin [U.S. Pat. No. 6,358,969] and 17-desmethylrapamycin [U.S. Pat. No. 6,670,168].

The term “desmethoxyrapamycin” or “desmethoxy” rapalog refers to a rapamycin or a rapalog core, in which a methoxy group (OMe) is missing. In one embodiment, the rapamycin excludes 41-desmethoxyrapamycin. With respect to formula (IIa), when R² is not the point of attachment to the linker, R² must be an O radical or OMe (i.e., the rapamycin is not 41-desmethoxyrapamycin).

In one embodiment, the esters and ethers of rapamycin are of the hydroxyl groups at the 42- and/or 31-positions of the rapamycin nucleus, esters and ethers of a hydroxyl group at the 27-position (following chemical reduction of the 27-ketone), and that the oximes, hydrazones, and hydroxylamines are of a ketone at the 42-position (following oxidation of the 42-hydroxyl group) and of 27-ketone of the rapamycin nucleus.

In another embodiment, 42- and/or 31-esters and ethers of rapamycin are described in the following patents: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. Nos. 5,118,678; 5,411,967; 5,480,989; 5,480,988; 5,489,680); amino carbamate esters (U.S. Pat. No. 5,463,048); silyl ethers (U.S. Pat. No. 5,120,842); aminoesters (U.S. Pat. No. 5,130,307); acetals (U.S. Pat. No. 5,51,413); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462). The preparation of these esters and ethers is described in the patents listed above.

In yet another embodiment, 27-esters and ethers of rapamycin are described in U.S. Pat. No. 5,256,790. The preparation of these esters and ethers is described in the patent listed above.

In still another embodiment, oximes, hydrazones, and hydroxylamines of rapamycin are described in U.S. Pat. Nos. 5,373,014, 5,378,836, 5,023,264, and 5,563,145. The preparation of these oximes, hydrazones, and hydroxylamines is described in the above-listed patents. The preparation of 42-oxorapamycin is described in U.S. Pat. No. 5,023,263.

In another embodiment, rapamycins include rapamycin [U.S. Pat. No. 3,929,992], rapamycin 42-ester with 3-hydroxy-2-(hydroxymethyl)-2-methylpropionic acid [U.S. Pat. No. 5,362,718], 42-Q-(2-hydroxy)ethyl rapamycin [U.S. Pat. No. 5,665,772], and 42-epi-tetrazolyl rapamycin [2006/0198870 A1]. The preparation and use of hydroxyesters of rapamycin, including CCI-779, is described in U.S. Pat. Nos. 5,362,718 and 6,277,983. In one embodiment, 42-esters with dicarboxylic acids, such as 42-hemisuccinate, 42-hemiglutarate and 42-hemiadipates, and the 42-ester of formula (IIb) are used for the synthesis of the conjugates.

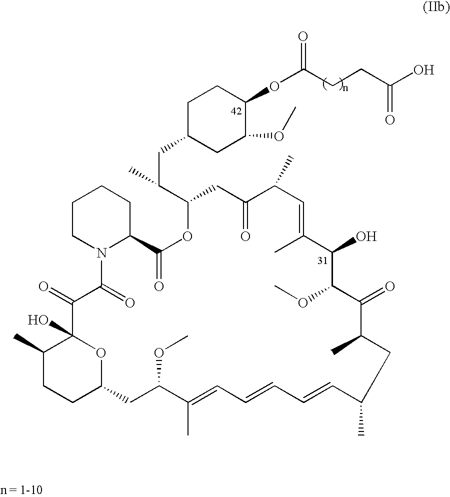

In another aspect, an mTOR inhibitor-L-wortmannin complex is provided. As used herein, the term “mTOR inhibitor” refers to a compound or ligand, or a pharmaceutically acceptable salt thereof, that inhibits cell replication by blocking the progression of the cell cycle from G1 to S. The term includes the neutral tricyclic compound rapamycin (sirolimus) and other rapamycin compounds, including, e.g., rapamycin derivatives, rapamycin analogues, other macrolide compounds that inhibit mTOR activity, and all compounds included within the definition below of the term “a rapamycin”. In one embodiment, the mTOR inhibitor is a rapamycin as defined herein.

In another embodiment, an FK-506-L-wortmannin complex is provided. For example, such a complex may utilize 32-esters of FK-506 (formula A) from an FK-506 compound having the structure of formula (III) illustrated below.

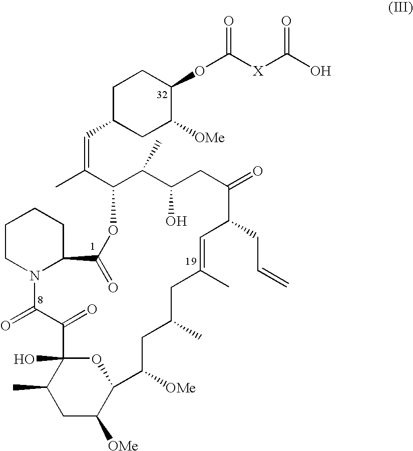

In another embodiment, an mTOR inhibitor-L-wort conjugate is described, provided that FK-506 compounds are excluded from the conjugates described herein.

In yet another embodiment, a rapamycin-L-wortmannin is described which excludes 41-desmethoxyrapamycins.

In still another embodiment, an mTOR inhibitor-L-wortmannin is described which excludes rapamycins of the structure:

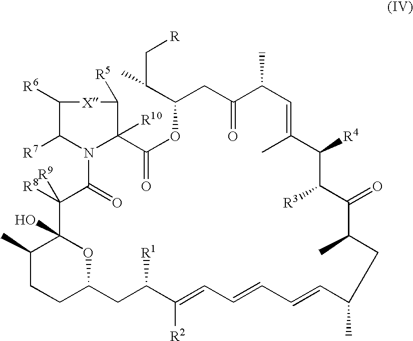

wherein, R¹ is selected from among OH, an ester, an ether, and a point of attachment to L, which L may be bound to the core through one of the preceding groups; R² is methyl or H; R³ is selected from among H, OH, an ester, an ether, and a point of attachment to the linker, which linker may be bound to the core through one of the preceding groups; R⁴ is selected from among OH, an ester, an ether, an amide, a carbonate, a carbamate, a phosphate, and a point of attachment to the linker, which linker may be bound to the core through one of the preceding groups; R⁵, R⁶, and R⁷ are independently selected from among H, alkyl, halo, and hydroxyl; R⁸, R⁹ is H,H or ═O; R¹⁰ is selected from among H, alkyl, halo and hydroxyl; X″ is a bond or CHR¹¹; or —CHR⁵—X″—CHR⁶— is

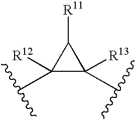

R¹¹, R¹², and R¹³ are independently selected from among H, alkyl, halo, and hydroxyl; R is selected from among:

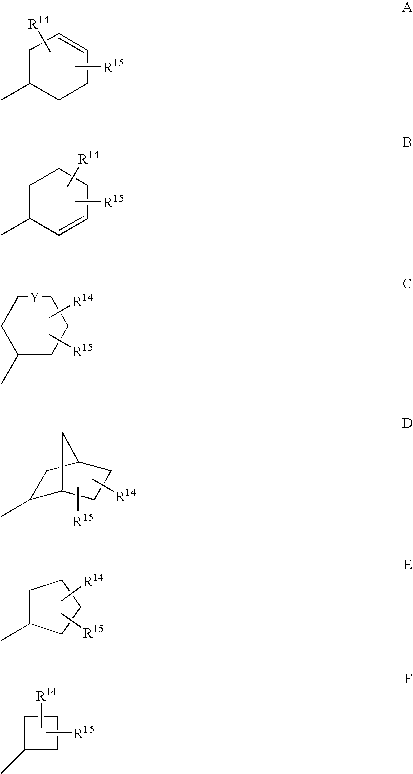

R¹⁴ and R¹⁵ are independently selected from among H, OH, halogen, thiol, amine, alkyl, an ester, an ether, an amide, a carbonate, a carbamate, a sulfonate, a phosphate, a tetrazole, and a point of attachment to the linker, which linker may be bound to the core through one of the preceding groups.

A wortmannin described herein refers to wortmannin and compounds which may be chemically or biologically modified as derivatives of the wortmannin nucleus, while retaining biological activity. Accordingly the term “a wortmannin” includes wortmannin and esters, ethers, oximes, hydrazones, and hydroxyamines of wortmannin, as well as wortmannins in which functional groups on the nucleus have been modified, for example through reduction or oxidation, a metabolite of wortmannin or a ring opened wortmannin. The term wortmannin also includes pharmaceutically acceptable salts of wortmannins, which are capable of forming such salts, either by virtue of containing an acidic or basic moiety. See, e.g., U.S. Pat. No. 5,378,725.

In one embodiment, a “wortmannin” is characterized by the class of compounds having the core structure of formula (Ia) provided below:

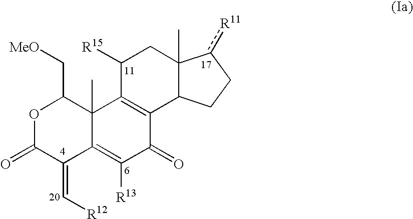

wherein, R¹¹ is selected from among O, OH, an ester, a carbonate, a carbamates, and an ether; R¹² and R¹³ are bound together via an O heteroatom; or R¹² is selected from among NR^{a}R^{b}, SR^{c}, and OR^{d}; R¹³ is selected from among OH, an ester, an ether, a carbonate, and a carbamate; R^{a}, R^{b}, R^{c}, and R^{d} are independently selected from among hydrogen, hydroxyl, alkyl, alkenyl, aryl, heterocyclic, and aralkyl; or R^{a} and R^{b} are optionally joined to form a ring; R¹⁵ is selected from among H, O, OH, an ester, a carbonate, and a carbamate.

In another embodiment, R¹¹ is O, R¹⁵ is OAc, and R¹² and R¹³ are bound together via an O heteroatom to form the wortmannin core.

In yet a further embodiment, R¹² is selected from among diethylamine, diallylamine, N,N,N′-trimethyl-1,3-propanediamine, piperidine, and N,N-dimethyl-N′-ethyl-ethylenediamine and R¹³ is —OH.

In a further embodiment, a “wortmannin” is characterized by the class of compounds having the core structure of formula (Ia1) provided below:

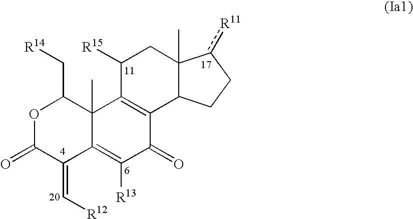

wherein, R¹¹ is selected from among O, OH, an ester, a carbonate, a carbamate, an ether, and a point of attachment to L; R¹² and R¹³ are bound together via an O heteroatom, or R¹² is selected from among an ester, an ether, a thioether, a thioester, an amino, and a point of attachment to L; R¹³ is selected from among OH, an ester, a carbonate, a carbamate, an ether, and a point of attachment to L; R¹⁴ is selected from among OH, an ester, an ether, and a point of attachment to L; R¹⁵ is selected from among O, OH, an ester, a carbonate, a carbamate, and a point of attachment to L; wherein at least one of R¹¹, R¹², R¹³, R¹⁴, and R¹⁵ is the point of attachment to L.

In still a further embodiment, L is bound to the wortmannin core through one of R¹¹, R¹², R¹³, R¹⁴, or R¹⁵.

In yet a further embodiment, R¹² is selected from among an ester, an ether, a thioether, a thioester, and a point of attachment to L. In another embodiment, R¹² is an amino. In a further embodiment, R¹² is an amino other than NH₂. In yet another embodiment, R¹² is NR^{a}R^{b}; R^{a} and R^{b} are independently selected from among H, alkyl, alkenyl, alkynyl, -(alkyl)-O-(alkyl)-, -(alkyl)-NR^{c}R^{d}—, -(alkyl)-C(═O)NR^{c}R^{d}—, cycloalkyl, aryl, and a heterocyclic group, with the proviso that both R^{a} and R^{b} cannot be H; or R^{a} and R^{b} may be taken together to form a three to seven membered heterocyclic ring having up to 3 heteroatoms which is optionally substituted by from 1 to 3 substituents independently selected from among halogen, hydroxyl, thio, alkyl, alkenyl, alkoxy, oxo, amino, cyano, C₁-C₃ perfluoroalkyl, alkylaryl, alkylheteroaryl, aryl, and heteroaryl; R^{c} and R^{d} are independently selected from among H, alkyl, alkenyl, alkynyl, aryl, cycloalkyl, and heterocycyl; or R^{c} and R^{d} are taken together to form a three to seven membered cyclic or heterocyclic ring having up to 3 heteroatoms which is optionally substituted by 1 to 3 substituents independently selected from among halogen, hydroxyl, thio, alkyl, alkenyl, alkoxy, oxo, amino, cyano and C₁-C₃ perfluoroalkyl. In a further embodiment, R^{a} is H and R^{b} is phenyl or R^{a} and R^{b} are a lower alkyl. In still a further embodiment, R¹¹ is O. In yet another embodiment, R¹² and R¹³ are bound together via an O heteroatom.

In one embodiment, the wortmannin may be a ring-opened wortmannin in which the furan ring is opened, i.e., R¹² and R¹³ are independent substituents. Nucleophilic addition to the electrophilic C-20 position of wortmannin results in a wortmannin derivative in which the furan ring is opened. Such ring-opened compounds are described (Wipf, Peter, et al. (2004) “Synthesis and biological evaluation of synthetic viridins derived from C(20)-heteroalkylation of the steroidal PI-3-kinase inhibitor wortmannin,” Org. Biomol. Chem., 2, 1911-1920); US Patent Application Publication Nos. 2003/0109572 to Powis and 2006/0128793 (application Ser. No. 11/248,510, filed Oct. 10, 2005).

In another embodiment, the wortmannin derivative is a 17-hydroxywortmannin. 17-Hydroxywortmannins may be prepared by the reduction of wortmannin, for example with diborane. 17-Hydroxywortmannins and other derivatives may be prepared according to US Patent Application Publication Nos. 2004/0213757 and 2006/0128793. Further wortmannin derivatives may be derived form the acetylation of the C-17 hydroxyl group. 17-hydroxywortmannin can be treated with a nucleophile such a as an amine to give a furan ring opened compound. 17-hydroxywortmannin can also be formylated at the 17-position then treated with a nucleophile to give a furan ring opened compound.

In another embodiment, the wortmannin derivative is 11-O-desacetylwortmannin. 11-O-desacetylwortmannin may be prepared by the literature procedure (Creemer C. L., et al (1996), “Synthesis and in vitro Evaluation of New Wortmannin Esters: Potent Inhibitors of Phosphatidylinositol 3-Kinase”, J. Med. Chem. 39, 5021-5024). Further 11-O-desacetylwortmannin derivatives are furan ring opened compounds with nucleophiles.

In another embodiment, the wortmannin may be conjugated to a water-soluble polymer such as PEG and as described in US Patent Application Publication Nos. 2004/0213757 and 2006/0128793 (U.S. patent application Ser. No. 11/248,510, filed Oct. 13, 2005).

The biosynthetic production of wortmannin is known in the art and the derivatives are synthesized from wortmannin.

### Conjugates

A conjugate is formed by linking a rapamycin and a wortmannin via a linker. Suitably, following administration of a conjugate to a subject, the linker is removed in whole or part from one or both of the rapamycin or the wortmannin.

The linker may be removed by any process without limitation, e.g., hydrolysis, enzymatic, pH, etc. In one embodiment, the linker is hydrolysable. In another embodiment, the linker is enzymatically cleaved. The term “hydrolysed” or “hydrolysable” and “enzymatically cleaved” or “enzymatically cleaveable” as used herein refers to the mechanism by which the linker group released in vivo.

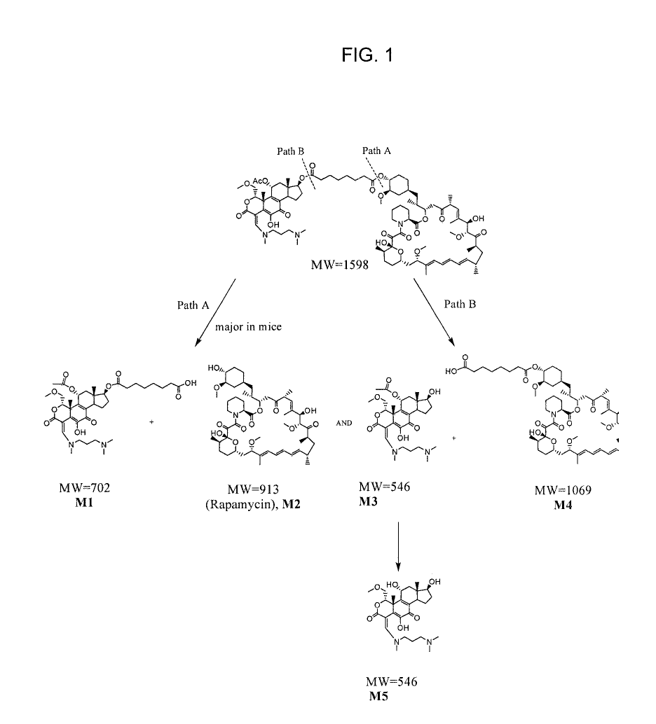

The linker may be completely removed from one or both of its binding partners (i.e., the rapamycin or the wortmannin). In such an embodiment, no member of the linker group remains bound to the rapamycin or the wortmannin following its removal. In another embodiment, the linker is partially removed from one or both of its binding partners. In this embodiment, the linker is cleaved such that the rapamycin and the wortmannin are separated; however, some part of the linker remains bound to the rapamycin or wortmannin. In one embodiment, a composition comprising an effective amount of conjugates may be processed in vivo, such that the conjugates afford a mixture of partially and completely removed linker—rapamycin and/or partially and completely removed linker—wortmannin metabolites. See, e.g., FIG. 1, which illustrates the metabolic pathways for an exemplary conjugate in a mammalian subject.

In one embodiment, the linker is characterized by formula (V):

-Z¹-X-Z²- (V)

wherein, Z¹ and Z² are independently selected from among a bond, O, —N(R⁰)—, S, —OC(═O)—, —OC(═O)O—, —N(R⁰)C(═O)—, —OC(═O)N(R⁰)—, —N(R⁰)C(═O)N(R⁰)—, —OC(═S)N(R⁰)—, —N(R⁰)C(═S)N(R⁰)—, and ═N—N(R⁰)—; R⁰ is at each occurrence independently selected from among H, alkyl, alkenyl, and aryl; and X is selected from among a hydrocarbon chain having from 1 to 16 carbon atoms which may be branched or unbranched, saturated or unsaturated, and optionally substituted with one or more of oxy, amine, sulfide, alkyl, alkenyl, aryl, alkoxy, hydroxyl, and halogen and/or interrupted by one or more ether (—O—), amine (—NH—), sulfide (—S—), —S(O)ₙ—, —N(R⁰)—, —C(═O)N(R⁰)—, or —OC(═O)N(R⁰)— and n is 0 to 2. X may also be selected from among cycloalkyl, aryl, alkylarylalkyl, heteroaryl and a heterocyclic group. In a further embodiment, Z¹ and Z² are independently a bond, i.e., L may be -Z¹-X—, —X—, or —X-Z².

In one embodiment, the conjugate excludes peroxide (O—O), O—N, and O—S bonds between the rapalog or wort core and linker. Thus, where the linker contains a terminal O, N or S group, the mTOR/rapamycin or wort core does not permit an O to be bound to the group.

As used herein, the term “alkyl” refers to both straight- and branched-chain saturated aliphatic hydrocarbon groups. In one embodiment, an alkyl group has 1 to about 16 carbon atoms. In another embodiment, an alkyl group has 1 to 10 carbon atoms or 1 to 8 carbon atoms (i.e., C₁, C₂, C₃, C₄, C₅ C₆, C₇, or C₈). An alkyl group having 1 to about 6 carbon atoms (i.e., C₁, C₂, C₃, C₄, C₅ or C₆) may be referred to as a “lower alkyl” group. In a further embodiment, an alkyl group has 1 to about 4 carbon atoms (i.e., C₁, C₂, C₃, or C₄). Particularly desirable alkyl groups include methyl, ethyl, propyl, isopropyl, butyl, isobutyl, and tert-butyl. Unless other substituents are specified, the alkyl group may be optionally substituted with one or more substituents selected from halo, CN, CO₂R, C(O)R, C(O)NR₂, NR₂, NO₂, and OR. Other suitable substituents are described herein in the definition of “substituted alkyl”.

The term “alkylarylalkyl” or “alkylaralkyl” refers to an alkyl group which is substituted with an aryl group which is itself substituted with an alkyl group.

The term “aryl” as used herein refers to an aromatic, carbocyclic system, e.g., of about 4 to 14 carbon atoms, which can include a single ring or multiple aromatic rings fused or linked together where at least one part of the fused or linked rings forms the conjugated aromatic system. The aryl groups include, but are not limited to, phenyl, naphthyl, biphenyl, anthryl, tetrahydronaphthyl, phenanthryl, indene, benzonaphthyl, and fluorenyl.

The term “cycloalkyl” is used herein to refer to cyclic, saturated aliphatic hydrocarbon groups. In one embodiment, a cycloalkyl group has 3 to about 8 carbon atoms (i.e., C₃, C₄, C₅, C₆, C₇, or C₈). In another embodiment, a cycloalkyl group has 3 to about 6 carbon atoms (i.e., C₃, C₄, C₅ or C₆).

The term “alkoxy” as used herein refers to the O(alkyl) group, where the point of attachment is through the oxygen-atom and the alkyl group can be substituted as noted above.

The term halo or halogen refers to elemental Cl, Br, F, or I or a group containing same.

The term “heterocycle” or “heterocyclic” as used herein can be used interchangeably to refer to a stable, saturated or partially unsaturated 3- to 9-membered monocyclic or multicyclic heterocyclic ring. The heterocyclic ring has in its backbone carbon atoms and one or more heteroatoms including nitrogen, oxygen, and sulfur atoms. In one embodiment, the heterocyclic ring has 1 to about 4 heteroatoms in the backbone of the ring. When the heterocyclic ring contains nitrogen or sulfur atoms in the backbone of the ring, the nitrogen or sulfur atoms can be oxidized. The term “heterocycle” or “heterocyclic” also refers to multicyclic rings in which a heterocyclic ring is fused to an aryl ring of about 6 to about 14 carbon atoms. The heterocyclic ring can be attached to the aryl ring through a heteroatom or carbon atom provided the resultant heterocyclic ring structure is chemically stable. In one embodiment, the heterocyclic ring includes multicyclic systems having 1 to 5 rings.

A variety of heterocyclic groups are known in the art and include, without limitation, oxygen-containing rings, nitrogen-containing rings, sulfur-containing rings, mixed heteroatom-containing rings, fused heteroatom containing rings, and combinations thereof. Examples of heterocyclic groups include, without limitation, tetrahydrofuranyl, piperidinyl, 2-oxopiperidinyl, pyrrolidinyl, morpholinyl, thiamorpholinyl, thiamorpholinyl sulfoxide, pyranyl, pyronyl, dioxinyl, piperazinyl, dithiolyl, oxathiolyl, dioxazolyl, oxathiazolyl, oxazinyl, oxathiazinyl, benzopyranyl, benzoxazinyl and xanthenyl.

The term “heteroaryl” as used herein refers to a stable, aromatic 5- to 14-membered monocyclic or multicyclic heteroatom-containing ring. The heteroaryl ring has in its backbone carbon atoms and one or more heteroatoms including nitrogen, oxygen, and sulfur atoms. In one embodiment, the heteroaryl ring contains 1 to about 4 heteroatoms in the backbone of the ring. When the heteroaryl ring contains nitrogen or sulfur atoms in the backbone of the ring, the nitrogen or sulfur atoms can be oxidized. The term “heteroaryl” also refers to multicyclic rings in which a heteroaryl ring is fused to an aryl ring. The heteroaryl ring can be attached to the aryl ring through a heteroatom or carbon atom provided the resultant heterocyclic ring structure is chemically stable. In one embodiment, the heteroaryl ring includes multicyclic systems having 1 to 5 rings.

A variety of heteroaryl groups are known in the art and include, without limitation, oxygen-containing rings, nitrogen-containing rings, sulfur-containing rings, mixed heteroatom-containing rings, fused heteroatom containing rings, and combinations thereof. Examples of heteroaryl groups include, without limitation, furyl, pyrrolyl, pyrazolyl, imidazolyl, triazolyl, pyridyl, pyridazinyl, pyrimidinyl, pyrazinyl, triazinyl, azepinyl, thienyl, dithiolyl, oxathiolyl, oxazolyl, thiazolyl, oxadiazolyl, oxatriazolyl, oxepinyl, thiepinyl, diazepinyl, benzofuranyl, thionapthene, indolyl, benzazolyl, purindinyl, pyranopyrrolyl, isoindazolyl, indoxazinyl, benzoxazolyl, quinolinyl, isoquinolinyl, benzodiazonyl, napthylridinyl, benzothienyl, pyridopyridinyl, acridinyl, carbazolyl, and purinyl rings.

The term “substituted heterocycle” and “substituted heteroaryl” as used herein refers to a heterocycle or heteroaryl group having one or more substituents including halogen, CN, OH, NO₂, amino, alkyl, cycloalkyl, alkenyl, alkynyl, C₁ to C₃ perfluoroalkyl, C₁ to C₃ perfluoroalkoxy, alkoxy, aryloxy, alkyloxy including —O—(C₁ to C₁₀ alkyl) or —O—(C₁ to C₁₀ substituted alkyl), alkylcarbonyl including —CO—(C₁ to C₁₀ alkyl) or —CO—(C₁ to C₁₀ substituted alkyl), alkylcarboxy including —COO—(C₁ to C₁₀ alkyl) or —COO—(C₁ to C₁₀ substituted alkyl), —C(NH₂)—N—OH, —SO₂—(C₁ to C₁₀ alkyl), —SO₂—(C₁ to C₁₀ substituted alkyl), —O—CH₂-aryl, alkylamino, arylthio, aryl, substituted aryl, heteroaryl, or substituted heteroaryl which groups may be optionally substituted. A substituted heterocycle or heteroaryl group may have 1, 2, 3, or 4 substituents.

The term “alkenyl” is used herein to refer to both straight- and branched-chain alkyl groups having one or more carbon-carbon double bonds. In one embodiment, an alkenyl group contains 2 to about 8 carbon atoms (i.e., C₂, C₃, C₄, C₅, C₆, C₇, or C₈). In another embodiment, an alkenyl groups has 1 or 2 carbon-carbon double bonds and 3 to about 6 carbon atoms (i.e., C₃, C₄, C₅ or C₆).

The term “alkynyl” is used herein to refer to both straight- and branched-chain alkyl groups having one or more carbon-carbon triple bonds. In one embodiment, an alkynyl group has 2 to about 8 carbon atoms (i.e., C₂, C₃, C₄, C₅, C₆, C₇, or C₈). In another embodiment, an alkynyl group contains 1 or 2 carbon-carbon triple bonds and 3 to about 6 carbon atoms (i.e., C₃, C₄, C₅, or C₆).

The terms “substituted alkyl”, “substituted alkenyl”, “substituted alkynyl”, and “substituted cycloalkyl” refer to alkyl, alkenyl, alkynyl, and cycloalkyl groups, respectively, having one or more substituents including, without limitation, hydrogen, halogen, CN, OH, NO₂, amino, aryl, heterocyclic groups, alkoxy, aryloxy, alkyloxy, alkylcarbonyl, alkylcarboxy, amino, and arylthio.

In one embodiment, Z¹ and Z² are independently selected from among a bond, O, —OC(═O)—, —OC(═O)O—, —OC(═O)N(R⁰)—, and OC(═S)N(R⁰)—.

In one embodiment, where the linker contains a terminal O, N or S group, the rapa or wort core does not provide for an O to be bound to this terminal O, N or S.

In one embodiment, Z¹ and Z² are a bond and X is an alkyl chain of 1 to 10 carbon atoms optionally substituted with one or more O groups.

In one embodiment, X is independently selected from among C₁-C₈ alkyl, C₂-C₈ alkenyl, cycloalkyl, aryl, and a heterocyclic group. In another embodiment, X is selected from among an alkyl chain of 1 to 16 carbon atoms interrupted by at least one group selected from among O, —S(O)ₙ—, —N(R⁰)—, —OC(═O)—, —OC(═O)O—, —C(═O)N(R⁰)—, and —OC(═O)N(R⁰)—, where n is 0 to 2. In one embodiment, X is (CH₂CH₂O)ₙ, where n is 1 to 8. In another embodiment, X is selected from among (CH₂)₂, (CH₂)₄, and (CH₂)₆. In a further embodiment, X is CH₂OCH₂.

In one embodiment, a rapamycin covalently linked with a wortmannin through a dicarboxylic acid linker of the formula is provided:

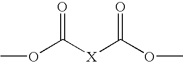

wherein X is as defined above. For example, X may be a hydrocarbon chain of the formula —(CH₂)ₙ—, where n is 1-16. Alternatively, X may be a hydrocarbon chain interrupted by an ether linkage, having the formula: —(CH₂)ₙ—O—(CH₂)ₙ—, where n is 1-16.

In one embodiment, a conjugate contains the rapamycin and the wortmannin in a ratio of 1:1, i.e., one rapamycin linked to one wortmannin.

In one embodiment, a rapamycin linked to a wortmannin has the structure of formula (IIa):

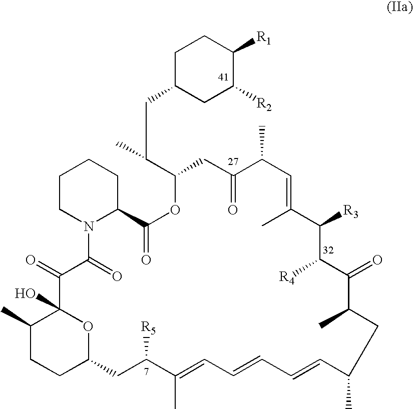

wherein:
the 42-position, i.e., R¹ is selected from among O, OH, an ester, an ether, an amide, a carbonate, a carbamate, a phosphate, a tetrazole, and a point of attachment to the linker, wherein the linker is optionally bound to the rapa core through the selected group;
the 41-position, i.e., R², is selected from among O, OH, an ester, an ether, and a point of attachment to the linker, wherein the linker is optionally bound to the rapa core through the selected group;
the 31-position, i.e., R³, is selected from among O, OH, an ester, an amide, a carbonate, a carbamate, an ether, and a point of attachment to the linker, wherein the linker is optionally bound to the rapa core through the selected group;
the 32-position, i.e., R⁴ is selected from among H, O, OH, an ester, an ether, and a point of attachment to the linker, wherein the linker is optionally bound to the rapa core through the selected group;
the 7-position, i.e., R⁵ is selected from among O, OH, an ester, an ether, and a point of attachment to the linker, wherein the linker is optionally bound to the rapa core through the selected group; and
at least one of R¹, R², R³, R⁴, and R⁵ is a point of attachment to the linker.

In one embodiment, the rapamycin excludes 41-desmethoxyrapamycin, i.e., where R² is H.

Examples of a variety of Rapamycin-L-wort formulae are provided below:

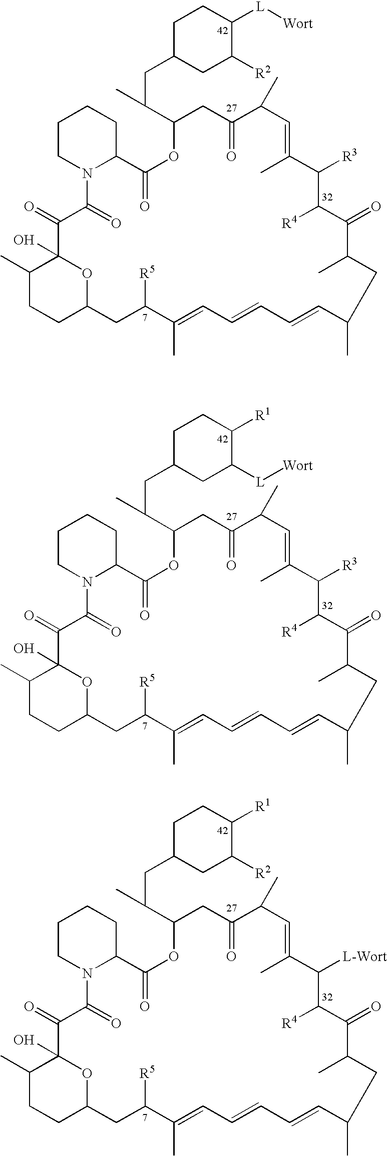

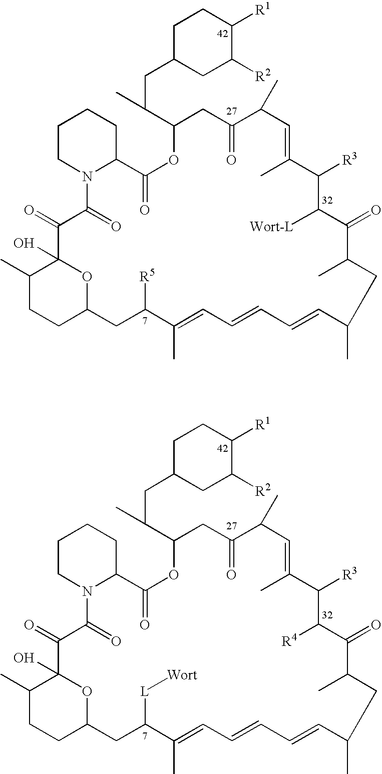

A linker may be bound independently to the rapamycin nucleus via any of R¹-R⁵ groups or through a bridging group. Such a bridging group may be independently selected from among an alkyl, an oxime, a hydrazone, a hydroxylamine, an ester, an ether, a thioester, and a thioether. In one embodiment, the bridging group is an ester at the 42 position, i.e., R¹.

In one embodiment, the rapamycin nucleus may be further substituted at any of R¹-R⁵ not bound to the linker, as described for the various rapamycin derivatives described above. For example, the rapamycin may be CCI-779, i.e., a rapamycin 42-ester with 3-hydroxy-2-(hydroxymethyl)-2-methylpropionic acid. In one embodiment, the linker is bound to the rapamycin nucleus through the 42-ester. In another embodiment, the linker is bound to the rapamycin nucleus through another position, e.g., R²-R⁵.

In one embodiment, the rapamycin used in the conjugate is rapamycin. In another embodiment, the rapamycin used in the used conjugate is a rapamycin 42-ester. In another embodiment, the rapamycin 42-ester is rapamycin 42-ester with 3-hydroxy-2-(hydroxymethyl)-2-methylpropionic acid. Still other suitable examples of rapamycins including, e.g. RAD001 (everolimus, Novartis), ABT478 (Abbott), and AP23573 [Ariad], will be readily apparent and can be readily selected from among the rapamycins described herein and known to those of skill in the art.

In one embodiment, the wortmannin has the core structure of formula (Ib):

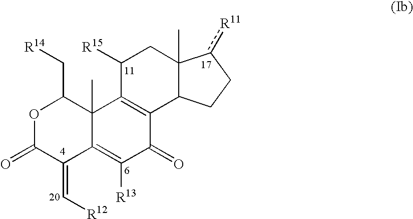

wherein, R¹¹ is selected from among O, OH, an ester, a carbonate, a carbamate, an ether, and a point of attachment to the linker, where the linker is optionally bound to the core through the selected group; R¹² and R¹³ are bound together via an O heteroatom; or R¹² is selected from among an ester, an ether, a thioether, a thioester, an amino, and the point of attachment to the linker, wherein the linker is optionally bound to the core through the selected group and R¹³ is selected from among OH, an ester, a carbonate, a carbamate, an ether, a thioether, and a point of attachment to the linker, wherein the linker is optionally bound to the core through the selected group; R¹⁴ is selected from among OH, an ester, an ether, and a point of attachment to the linker, wherein the linker is optionally bound to the core through the selected group; R¹⁵ is selected from among O, OH, an ester, a carbonate, a carbamate, and a point of attachment to the linker, wherein the linker is optionally bound to the core through the selected group; wherein at least one of R¹¹, R¹², R¹³, R¹⁴, and R¹⁵ is a point of attachment to the linker. A linker may be independently bound directly to any of the R¹¹-R¹⁵ groups or bound through a bridging group. Such a bridging group may be independently selected from among the groups recited above for R¹²-R¹⁵. In other embodiments, the bridging group may be selected from among an alkyl, an ester, an ether, a thioester, and a thioether.

In one embodiment, the wortmannin nucleus may be further substituted at any of R¹¹-R¹⁵ not bound to the linker, as described for the various wortmannin derivatives described above. For example, the wortmannin may be 17-hydroxywortmannin. In one embodiment, the linker is bound to the wortmannin nucleus through the 17-position. In another embodiment, a linker is bound to the 17-hydroxywortmannin through another position.

Examples of a variety of wortmannin-L-Rap formulae are provided below:

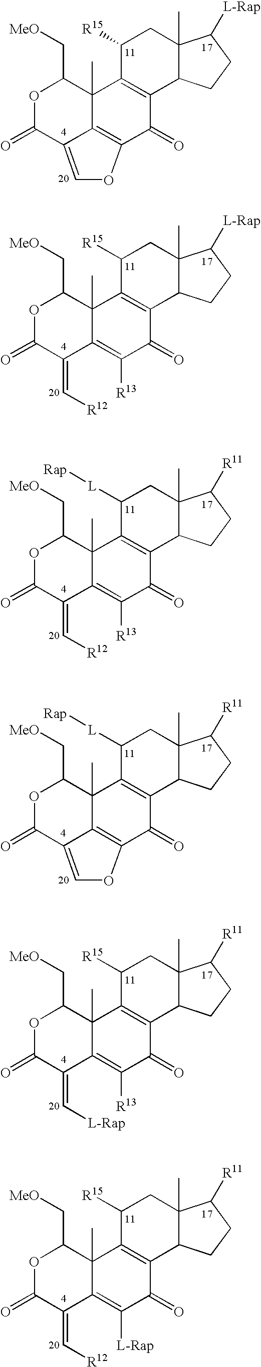

In one embodiment, R¹¹ is the point of attachment to the linker. In another embodiment, R¹¹ is an O.

In yet a further embodiment, R¹² is an amino group. In one embodiment, where R¹² is an amino group, it has the formula —NR^{a}R^{b}—, where R^{a} and R^{b} are independently selected from among H, alkyl, alkenyl, alkynyl, -(alkyl)-O-(alkyl)-, -(alkyl)-NR^{c}R^{d}—, -(alkyl)-C(═O)NR^{c}R^{d}—, cycloalkyl, aryl, heteroaryl, alkylaryl, alkylheteroaryl, and a heterocyclic group; or R^{a} and R^{b} may be taken together to form a three to seven membered heterocyclic ring having up to 3 heteroatoms which is optionally substituted by 1 to 3 substituents independently selected from among halogen, hydroxyl, thio, alkyl, alkenyl, alkoxy, oxo, amino, cyano, C₁-C₃ perfluoroalkyl, alkylaryl, alkylheteroaryl, aryl, and heteroaryl; R^{c} and R^{d} are independently selected from among H, alkyl, alkenyl, alkynyl, aryl, cycloalkyl, and heterocycyl; or R^{c} and R^{d} are taken together to form a three to seven membered cyclic or heterocyclic ring having up to 3 heteroatoms which is optionally substituted by 1 to 3 substituents independently selected from among halogen, hydroxyl, thio, alkyl, alkenyl, alkoxy, oxo, amino, cyano and C₁-C₃ perfluoroalkyl.

In one embodiment, R¹² has the formula —NHR^{a}—, where R^{a} is as defined above. In one embodiment, R^{a} is phenyl.

In another embodiment R^{a} and R^{b} are both lower alkyls.

In still another embodiment, R¹² and R¹³ are bound together via an O heteroatom.

Other suitable points of attachment to wortmannins will be readily apparent to one of skill in the art.

### Methods of Preparing the Conjugates

The compounds described herein are readily prepared by one of skill in the art according to the following schemes using commercially available starting materials or starting materials which can be prepared using literature procedures. These schemes show the preparation of conjugates in which “a rapamycin” is linked with “a wortmannin” through a di-ester linkage. While these schemes teach the principle of the present invention, with examples provided for the purpose of illustration, it will be understood that such conjugates through other types of functional group linkage such as amides, carbonates, carbamate, ethers, thio, hydrazones, et al, or through other linking positions are readily available by modification of, or additions to, procedures and protocols described herein. Variations on these methods, or other methods known in the art, can be readily performed by one of skill in the art given the information provided herein.

A synthesis of rapamycin-wortmannin conjugates through rapamycin 42-OH and wortmannin 17-OH positions via a di-ester linkage as described herein is outlined in Scheme 1. The conversion of wortmannin to 17-N, or 11-N substituted wortmannin analogs, and the synthesis of 42-N, 42-S substituted rapamycin analogs from rapamycin, can be readily performed by one of skill in the art, using such techniques as are described in, e.g., see “Comprehensive Organic Transformation” [Richard C. Larock, 2^{nd} edition, 1999] and others which are known in the art. See, also, rapamycin U.S. Pat. No. 5,527,907.

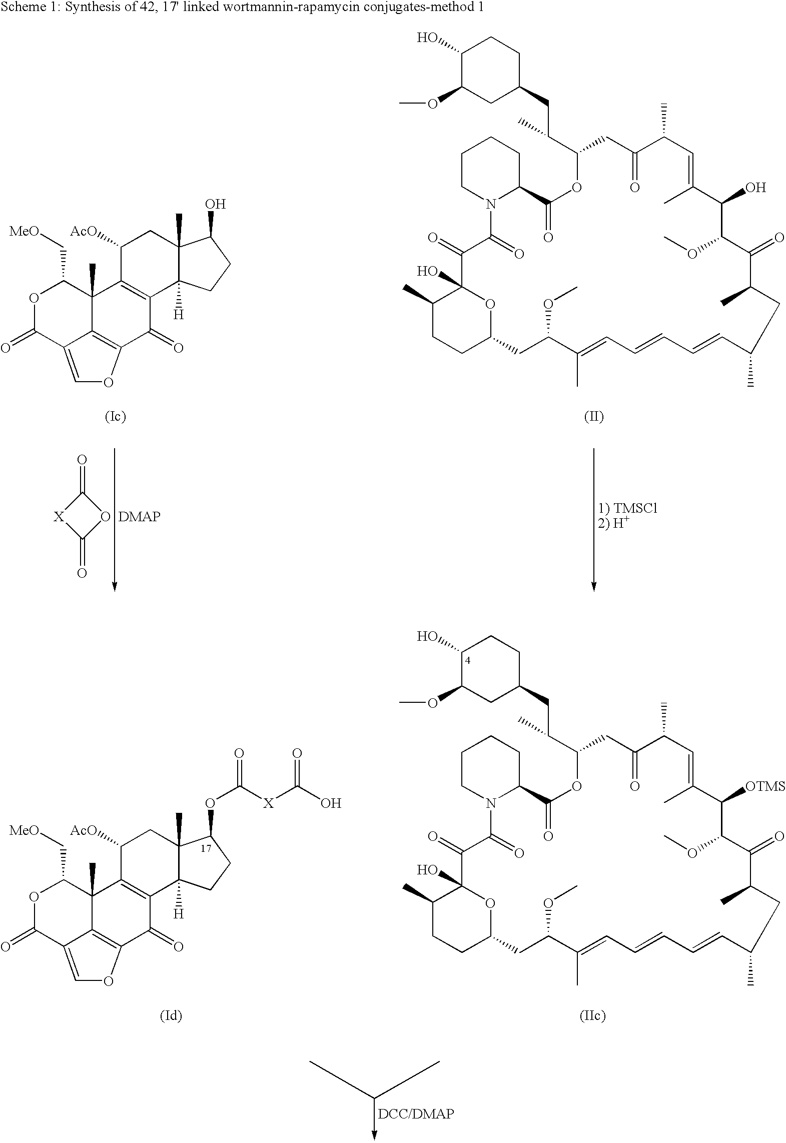

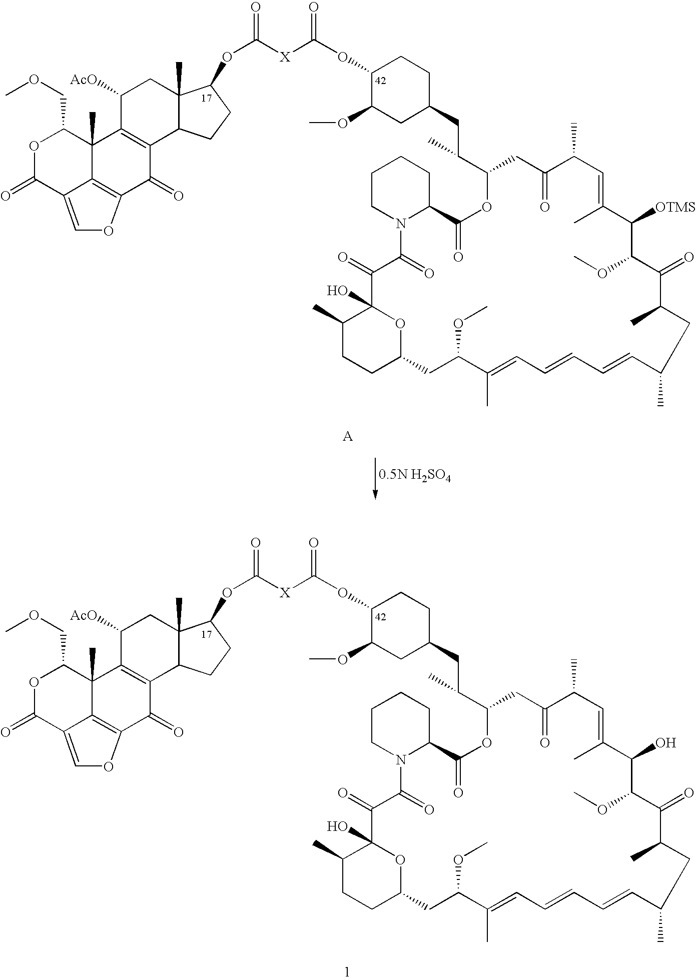

wherein X is selected from among a hydrocarbon chain having from 1 to 16 carbon atoms which may be branched or unbranched, saturated or unsaturated, and optionally substituted with one or more of amine, sulfide, alkyl, alkenyl, aryl, alkoxy, hydroxyl, and halogen; or may be interrupted by one or more ether (—O—), amine (—NH—) or sulfide (—S—) linkage, cycloalkyl, aryl, alkylarylalkyl, heteroaryl and a heterocyclic group.

A 17-hydroxywortmannin (Ic) is acylated with various cyclic anhydrides to give hemiacids (Id). These dicarboxylic monoesters are then coupled with rapamycin 31-trimethylsilyl ether (IIc) in the presence of a coupling reagent such as, e.g., N,N′-dicyclohexyl-carbodiimide (DCC), Diisopropylcarbodiimide (DIPC) or 1-Ethyl-3-[3-dimethylaminopropyl]-carbodiimide Hydrochloride (EDC) and a base such as 4-(dimethylamino)pyridine (DMAP), to give intermediates A, subsequent de-protection with diluted H₂SO₄ furnish desired 42,17′-linked wortmannin-rapamycin conjugate 1. Rapamycin 31-trimethylsilyl ether may be synthesized according to the procedure described in U.S. Pat. No. 6,277,983.

Alternatively, such di-ester linked wortmannin-rapamycin conjugates can be synthesized as described in Scheme 2. The dicarboxylic acid linker was first installed into rapamycin moiety via a lipase-catalyzed acylation method described in US Patent Application Publication No. 2005/0234087. These rapamycin hemiesters (IIb) were then coupled with 17-hydroxywortmannin under DCC/DMAP combination to give wortmannin-rapamycin conjugates in good yield.

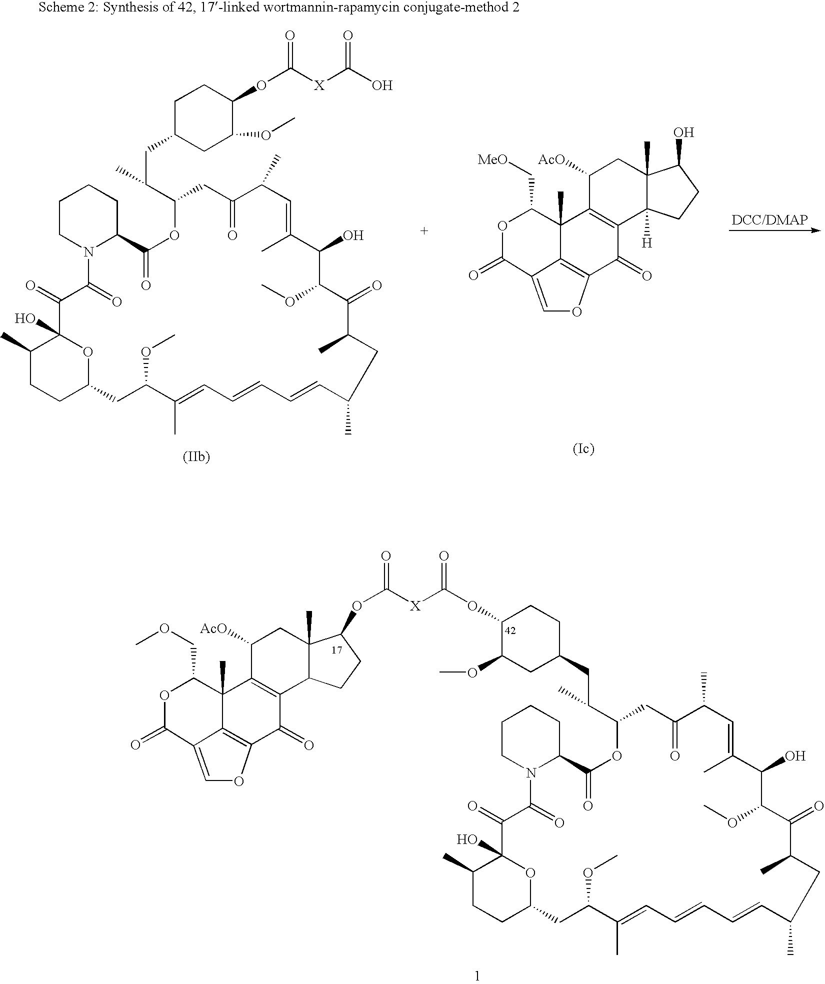

In one embodiment, X is selected from among (CH₂)₂, (CH₂)₄, and (CH₂)₆, or from among the substituents defined above.

In one embodiment, exemplary conjugates which can be readily prepared using the techniques described herein include, e.g., those having the structures:

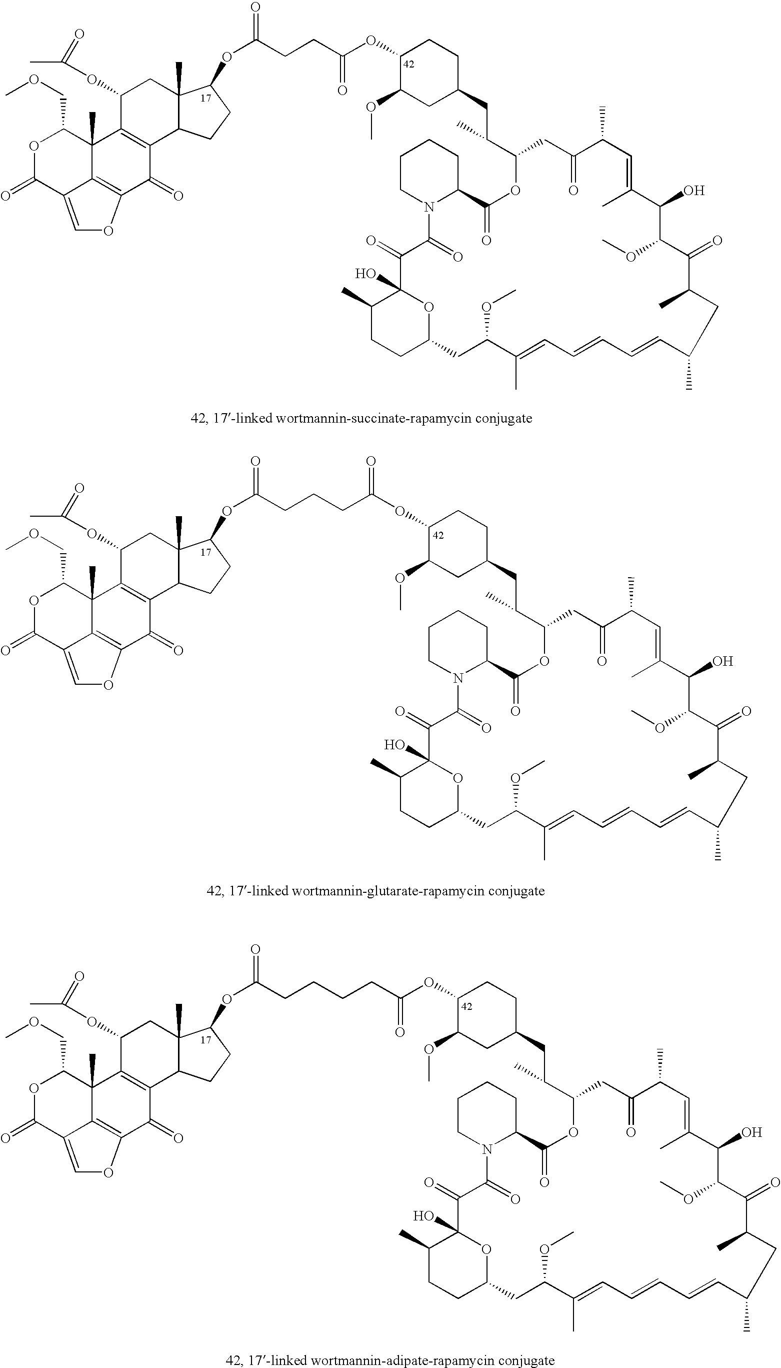

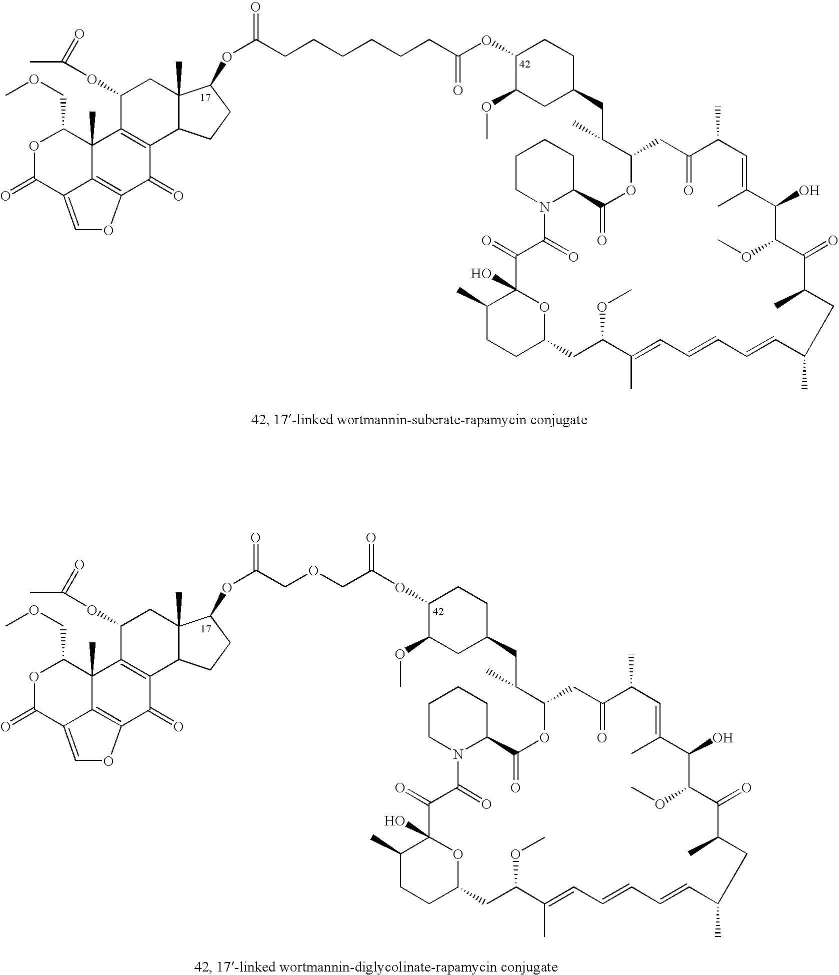

In one embodiment, these rapamycin-wortmannin conjugates compounds can be further converted to the furan ring-opened derivatives 2 with various R^{12′} containing nucleophiles such as thiols, amines, particularly secondary amines, and alcohols (Scheme 3). These nucleophiles can be selected from, for example, the list covered in US Published Patent Application No. 2006/0128793, published Jun. 15, 2006.

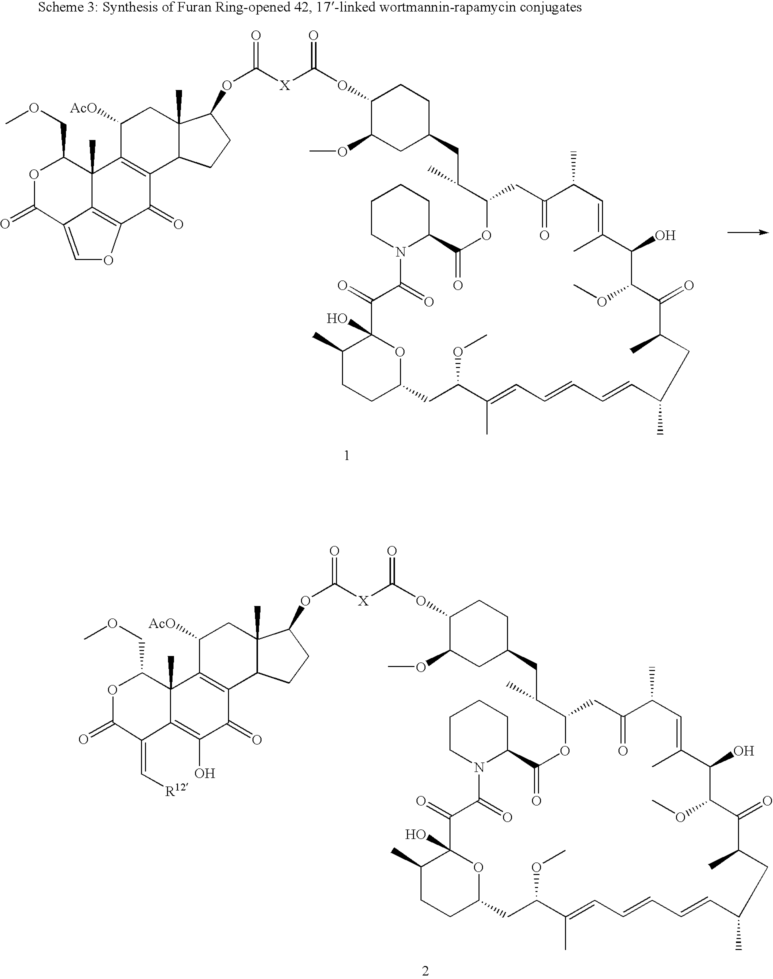

wherein, X is selected from among a hydrocarbon chain having from 1 to 16 carbon atoms which may be branched or unbranched, saturated or unsaturated, and optionally substituted with one or more of amine, sulfide, alkyl, alkenyl, aryl, alkoxy, hydroxyl, and halogen; or may be interrupted by one or more ether (—O—), amine (—NH—) or sulfide (—S—) linkage, cycloalkyl, aryl, alkylarylalkyl, and a heterocyclic group; R^{12′} is selected from among NR^{a}R^{b}, SR^{c}, and OR^{d}; R^{a} and R^{b} are independently selected from among H, alkyl, alkenyl, alkynyl, -(alkyl)-O-(alkyl)-, -(alkyl)-NR^{e}R^{f}, -(alkyl)-C(═O)NR^{e}R^{f}—, cycloalkyl, aryl, and a heterocyclic group; or taken together to form a three to seven membered heterocyclic ring having up to 3 heteroatoms which is optionally substituted by 1 to 3 substituents independently selected from among halogen, hydroxyl, thio, alkyl, alkenyl, alkoxy, oxo, amino, cyano, C₁-C₃ perfluoroalkyl, alkylaryl, alkylheteroaryl, aryl, and heteroaryl; R^{e} and R^{f} are independently selected from among H, alkyl, alkenyl, alkynyl, aryl, cycloalkyl, and heterocycyl; or taken together to form a three to seven membered cyclic or heterocyclic ring having up to 3 heteroatoms which is optionally substituted by 1 to 3 substituents independently selected from among halogen, hydroxyl, thio, alkyl, alkenyl, alkoxy, oxo, amino, cyano and C₁-C₃ perfluoroalkyl; R^{c} and R^{d} are independently selected from among H, alkyl, alkenyl, alkynyl, aryl, heteroaryl, cycloalkyl, and heterocycle, or R^{c} and R^{d} are taken together to form a three to seven membered cyclic or heterocyclic ring having up to 3 heteroatoms which is optionally substituted by 1 to 3 substituents independently selected from among halogen, hydroxyl, thio, alkyl, alkenyl, alkoxy, oxo, amino, cyano and C₁-C₃ perfluoroalkyl.

In one embodiment, exemplary conjugates in the form of amine adducts, i.e., furan ring in wortmannin portion was opened by various secondary amines, are illustrated below.

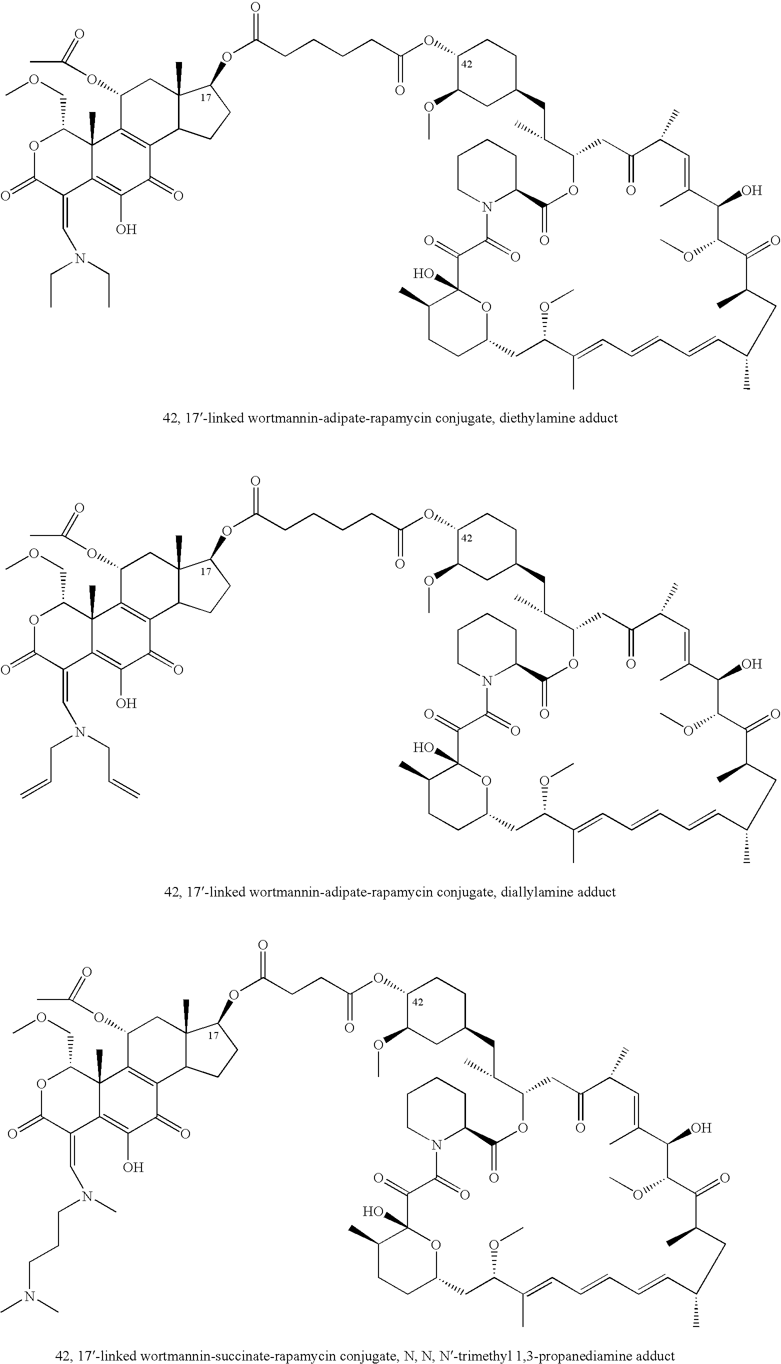

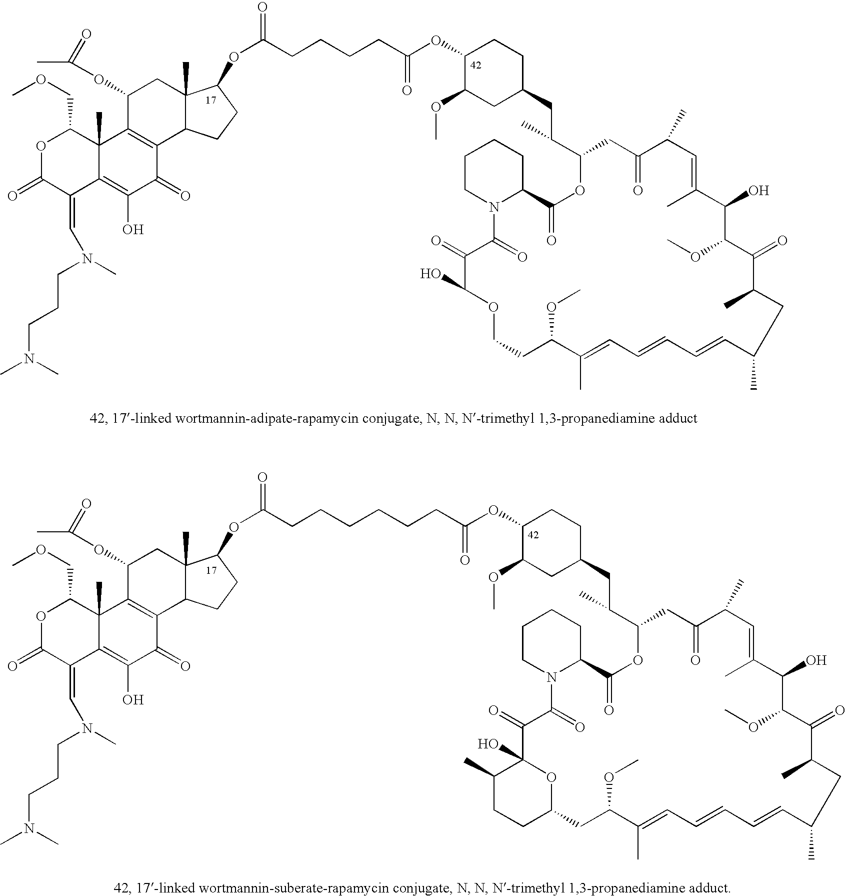

In another embodiment, synthesis of rapamycin-wortmannin conjugate through rapamycin 31-OH and wortmannin 17-OH positions via a di-ester linkage as described herein is outlined in Scheme 4. The wortmannin 17-dicarboxylic monoacid (Id) was coupled with rapamycin 42-TBS ether (IId) in the presence of a coupling reagent such as, e.g., DCC, DIPC or EDC and a base such as DMAP, to give intermediates B. Subsequent de-protection with diluted H₂SO₄ furnishes the desired 31,17′-linked wortmannin-rapamycin conjugate 3. Rapamycin 42-TBS ether may be synthesized according to the procedure described in European Patent No. 0507556A1.

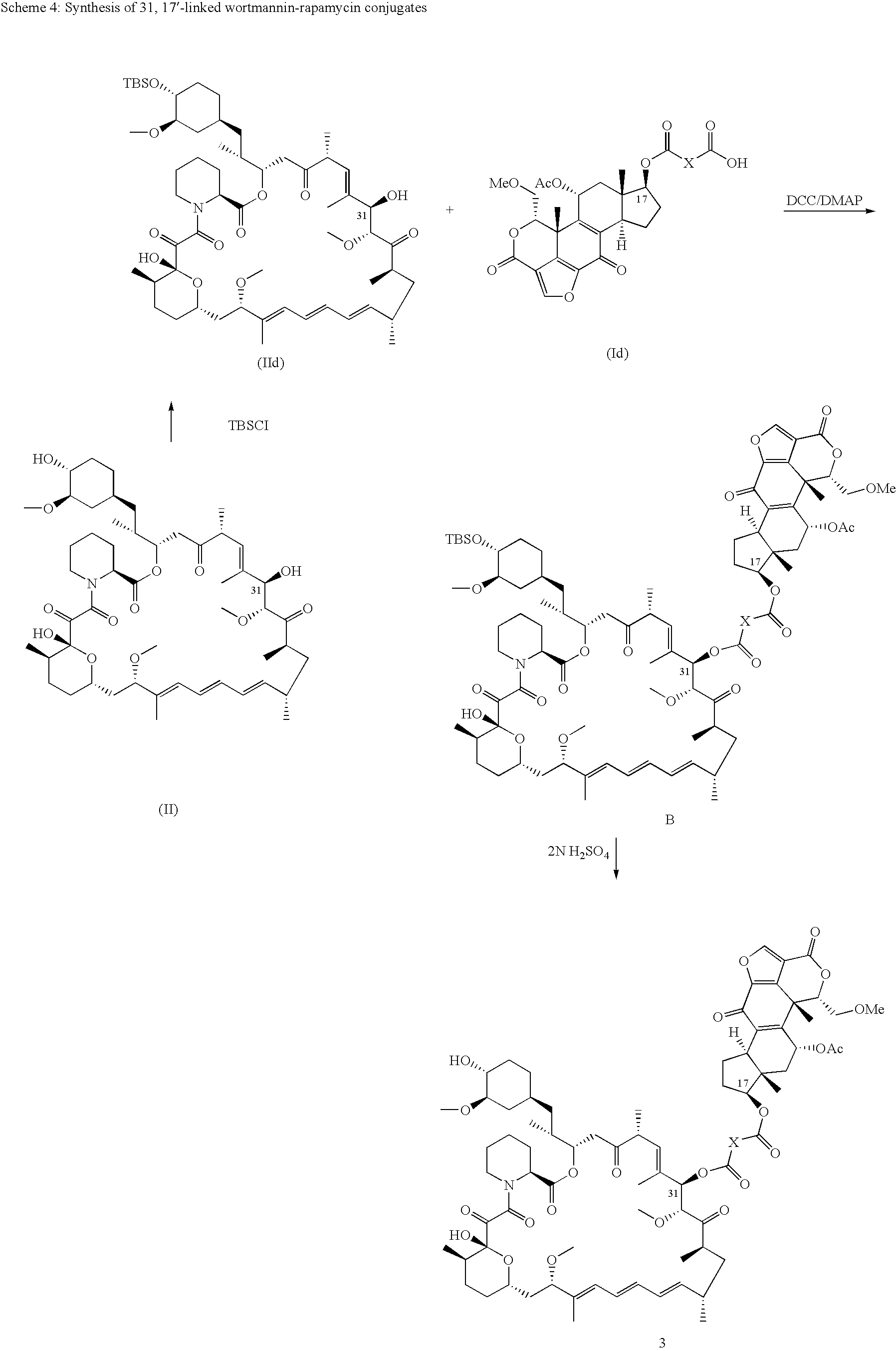

wherein, X is as defined herein.

Illustrative examples according to the above preparation include, but are not limited to the following structure

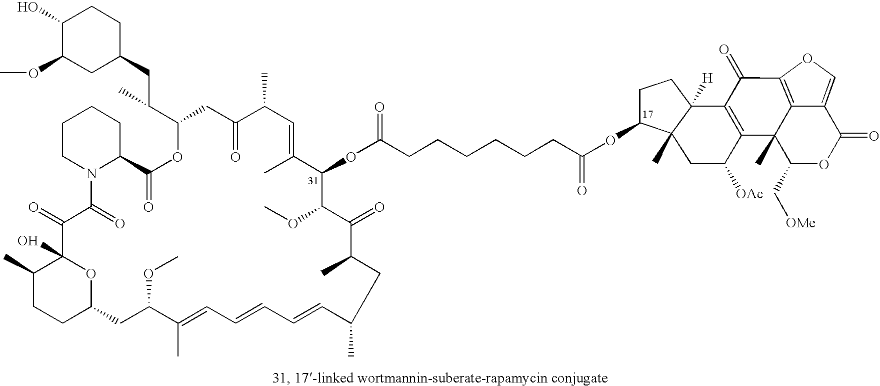

In one embodiment, 31,17′-linked wortmannin-rapamycin conjugate 3 can be treated with R^{12′} containing nucleophiles to give a furan ring opened conjugate 4 as depicted in Scheme 5.

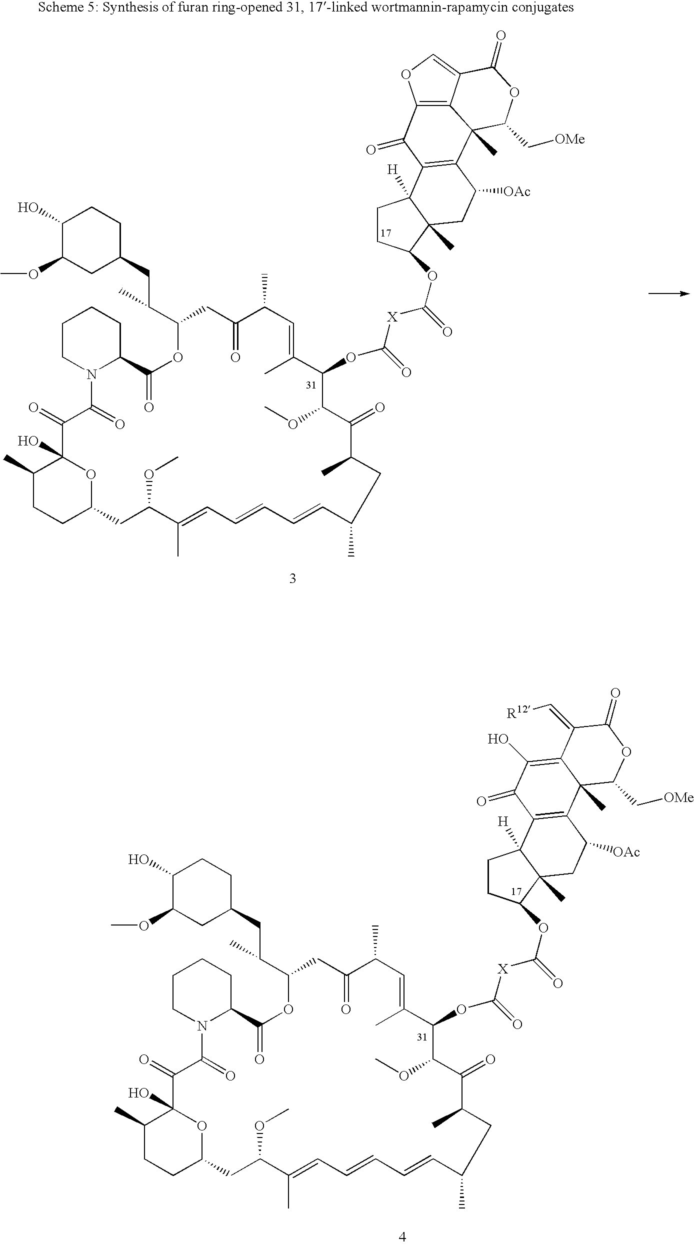

wherein, X and R^{12′} are as defined herein.

In one embodiment, the following exemplary compounds are provided:

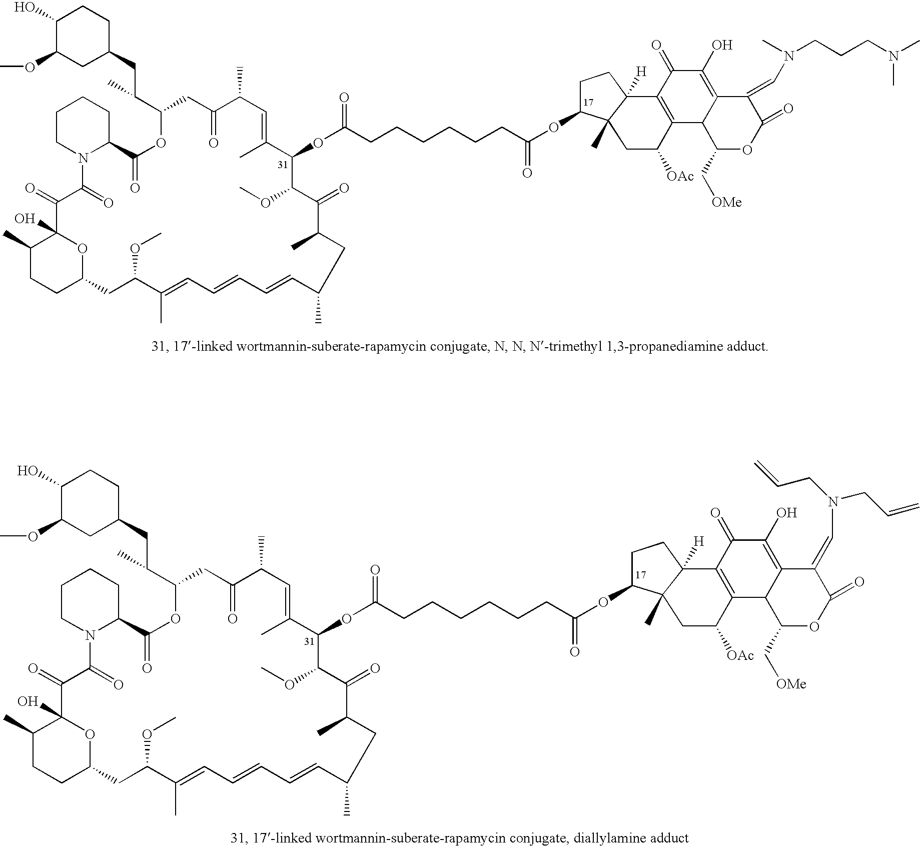

In still another embodiment, the conjugates can be prepared according to the Scheme 6 through the linking position of rapamycin 42-OH and wortmannin 11-OH. As for di-ester linked wortmannin-rapamycin, such conjugates (5) are readily available by coupling 11-desacetyl wortmannin 11-dicarboxylic monoacid (If) with rapamycin 31-TMS ether (IIc) in the presence of a coupling reagent such as, e.g. DCC, DIPC or EDC and a base such as DMAP, followed by de-protection with diluted H₂SO₄ in good overall yield.

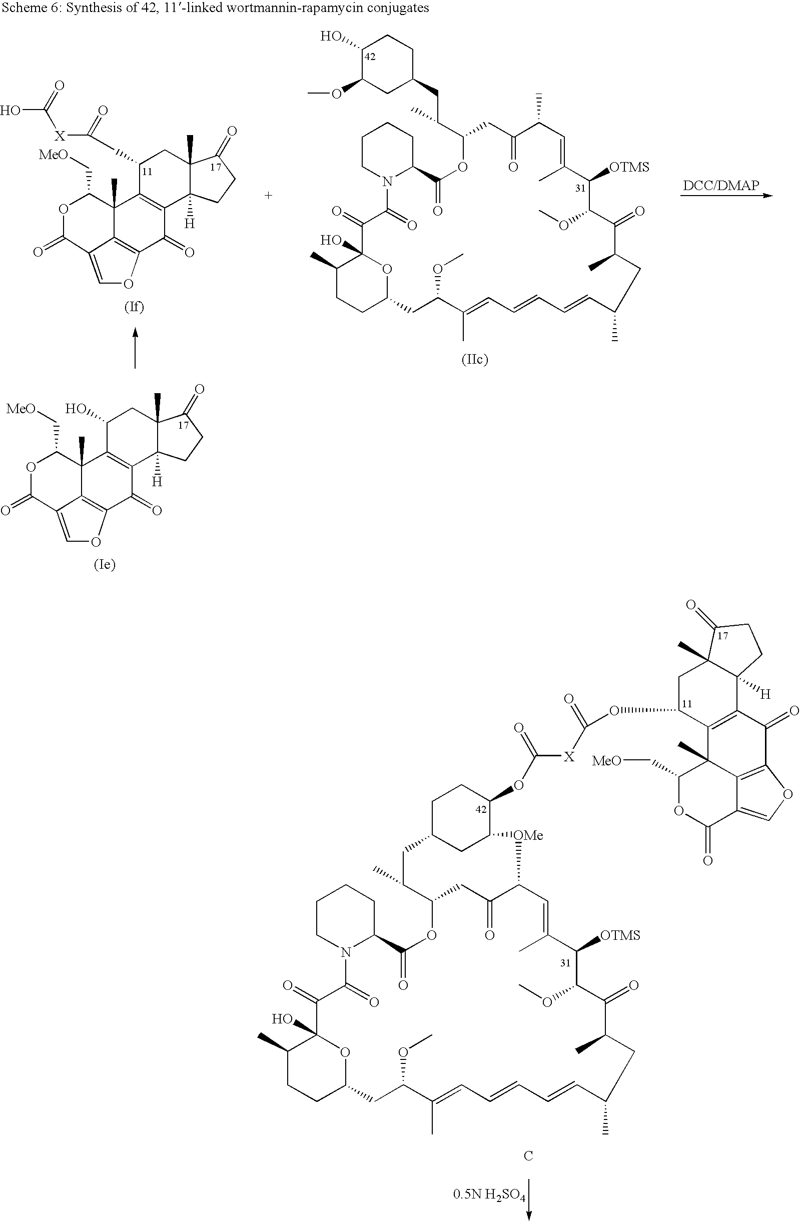

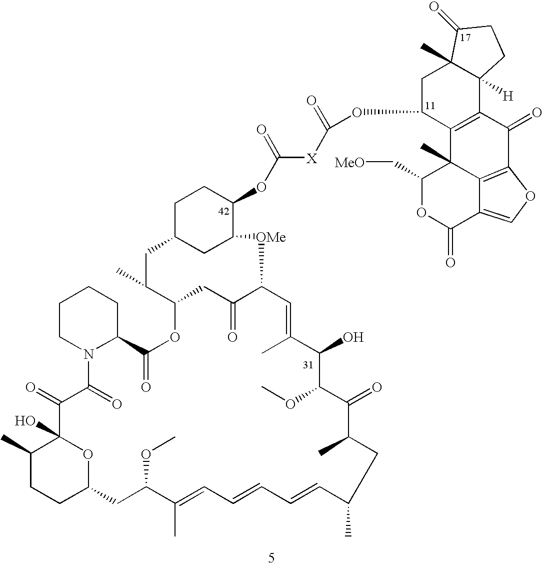

wherein, X is as defined herein.

An exemplary conjugate which can be readily prepared by employing procedures described above include, but is not limited to the structure:

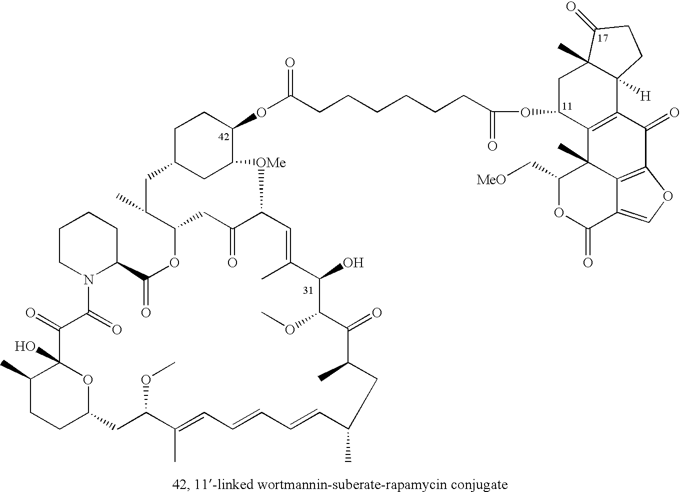

In one embodiment, such 42,11′-linked wortmannin-rapamycin conjugate 5 can be treated with R^{12′} containing nucleophiles to give furan ring opened conjugate 6 as depicted in scheme 7.

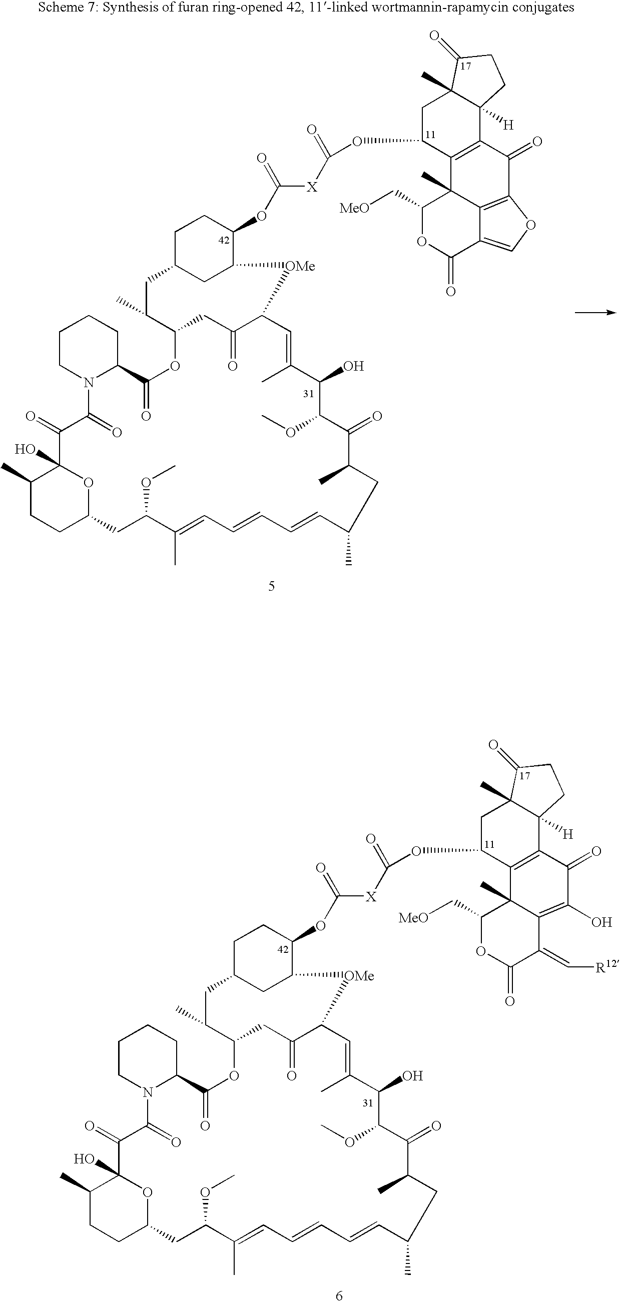

wherein, X and R^{12′} are as defined herein.

In one embodiment, the following exemplary compounds are provided:

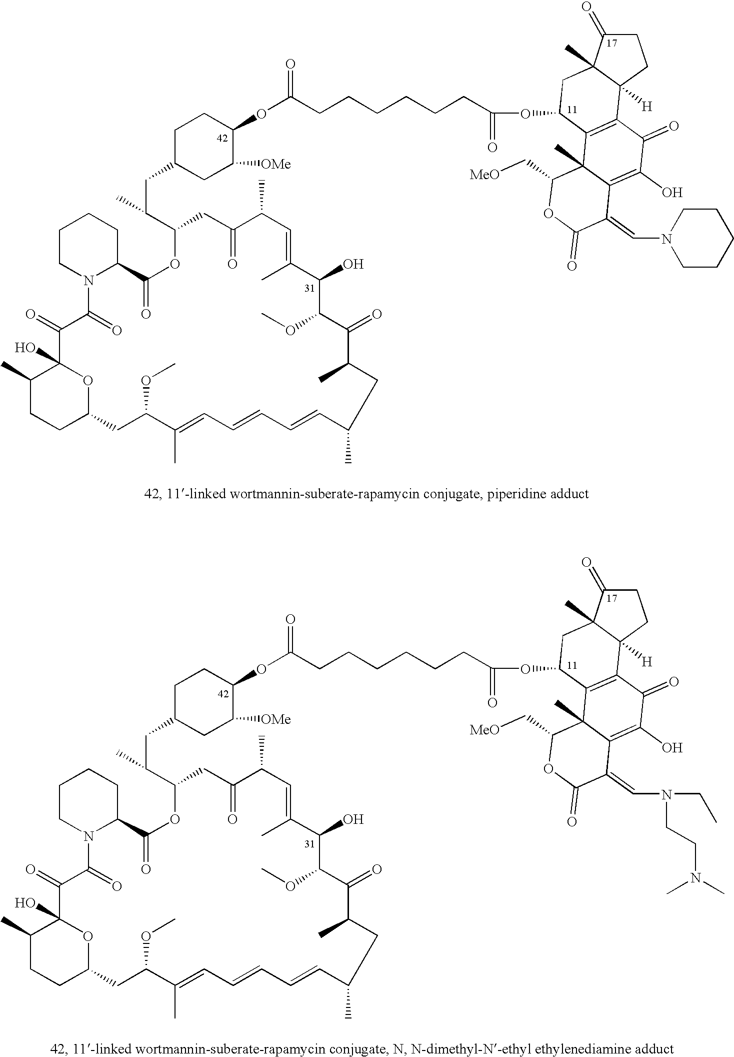

In yet still another embodiment, the conjugates can be prepared according to Scheme 8 through the linking position of rapamycin 31-OH and wortmannin 11-OH. As for di-ester linked wortmannin-rapamycin, such conjugates (7) are readily available by coupling 11-desacetyl wortmannin 11-dicarboxylic monoacid (If) with rapamycin 42-TBS ether (IId) in the presence of a coupling reagent such as, e.g., DCC, DIPC or EDC and a base such as DMAP, followed by de-protection (e.g., with diluted H₂SO₄) in excellent overall yield.

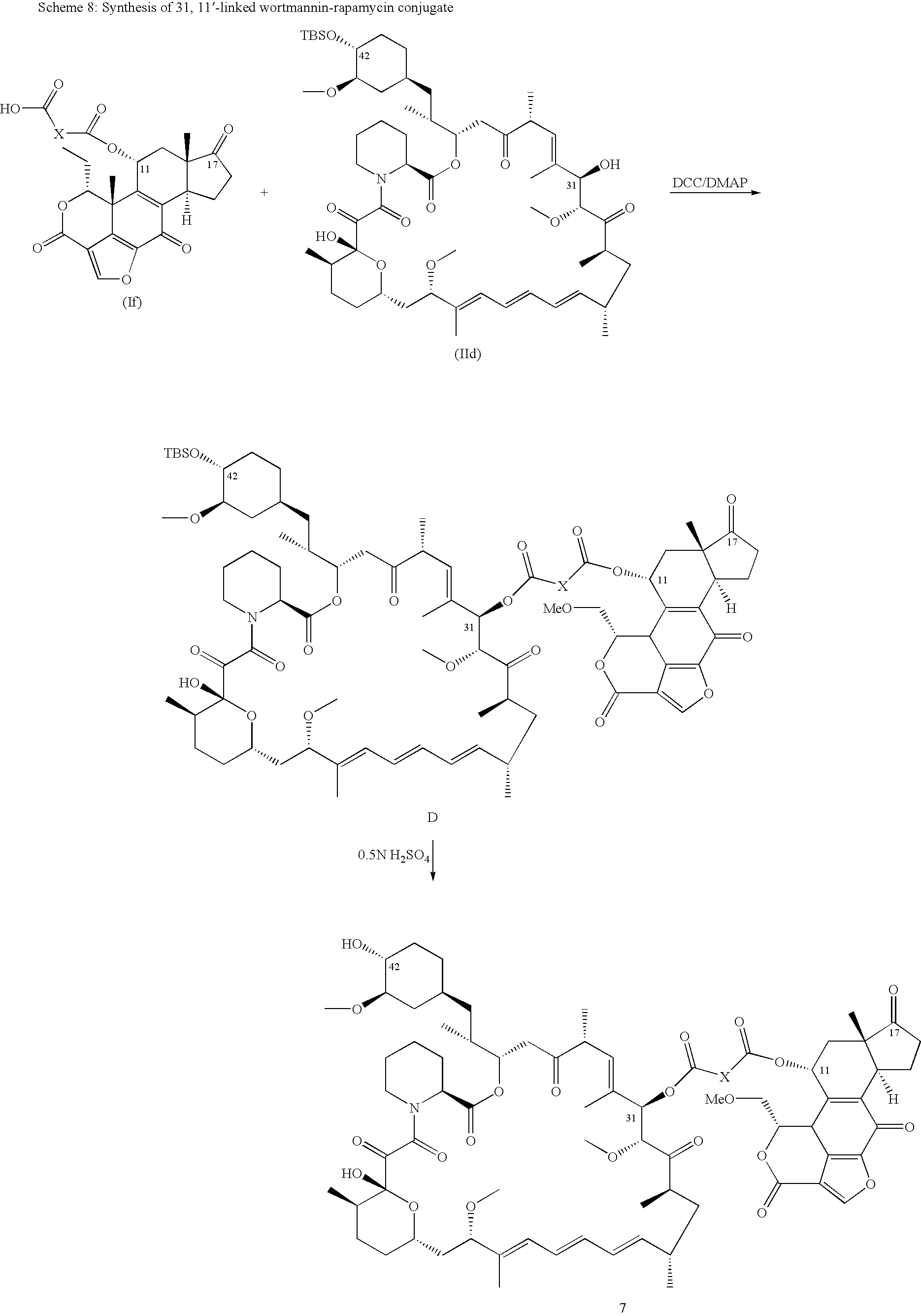

wherein, X is as defined herein.

An exemplary conjugate which can be readily prepared by employing procedures described above includes:

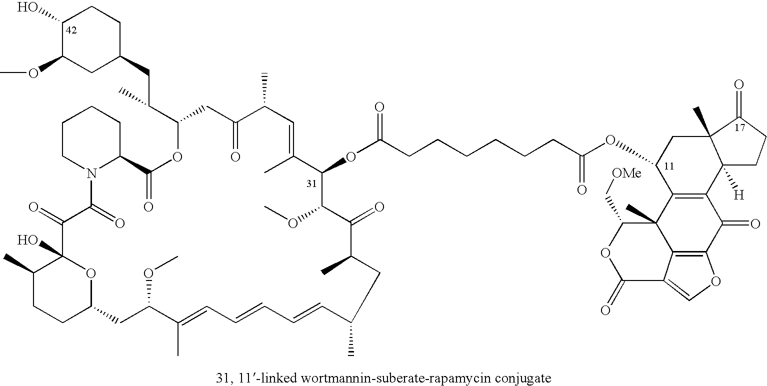

In one embodiment, such 31,11′-linked wortmannin-rapamycin conjugate 7 can be treated with R^{12′} containing nucleophiles to give furan ring opened conjugate 8 as depicted in scheme 9.

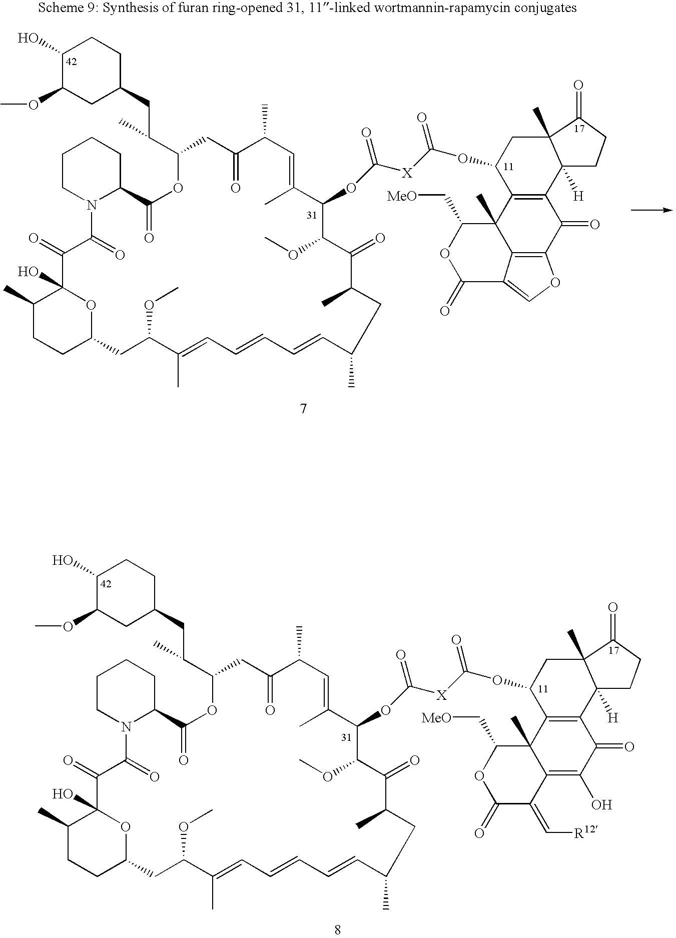

wherein, X and R^{12′} are as defined herein.

In one embodiment, the following exemplary compounds are provided:

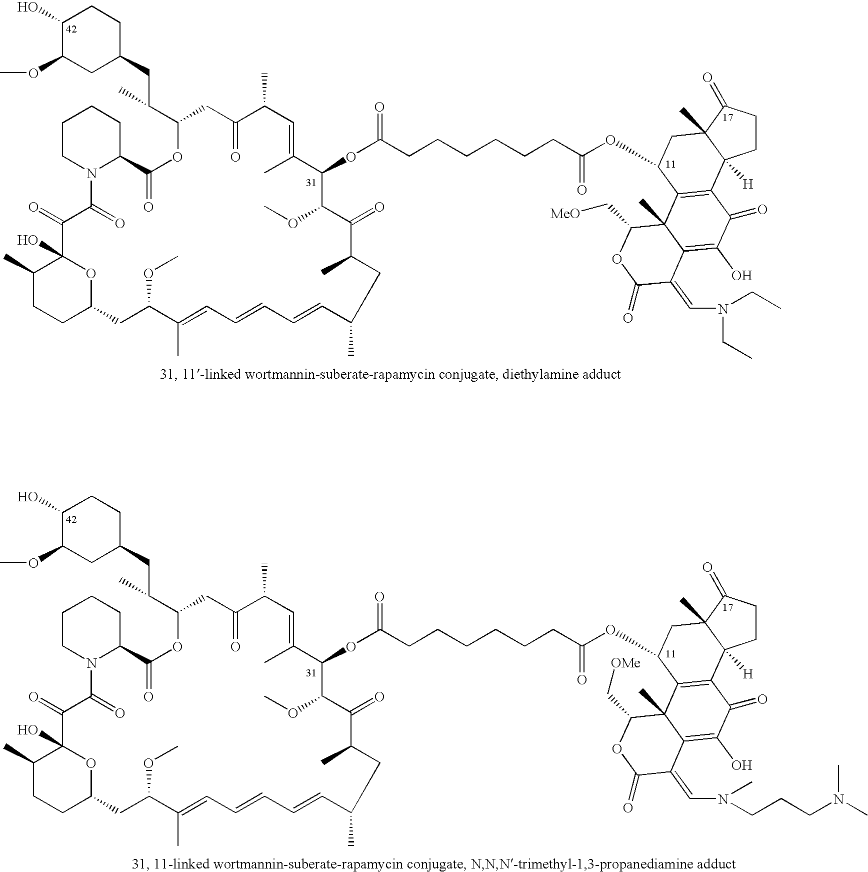

The presence of certain substituents in the conjugates may enable salts of the conjugates to be formed. Suitable salts include pharmaceutically or physiologically acceptable salts, for example acid addition salts derived from organic or inorganic acids, and salts derived from inorganic or organic bases. Acid addition salt including, e.g., acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids. Salts derived from inorganic and organic bases include alkali metal salts such as sodium, lithium, or potassium, magnesium, calcium and organic amine salts such as dimethylamine, diethylamine, morpholine, piperidine salts.

Particularly useful salts of the conjugates include pharmaceutically acceptable salts, especially acid addition pharmaceutically acceptable salts. An exemplary salt of the conjugate which can be readily prepared by employing procedures known in the skill of art include, but is not limited to the structure:

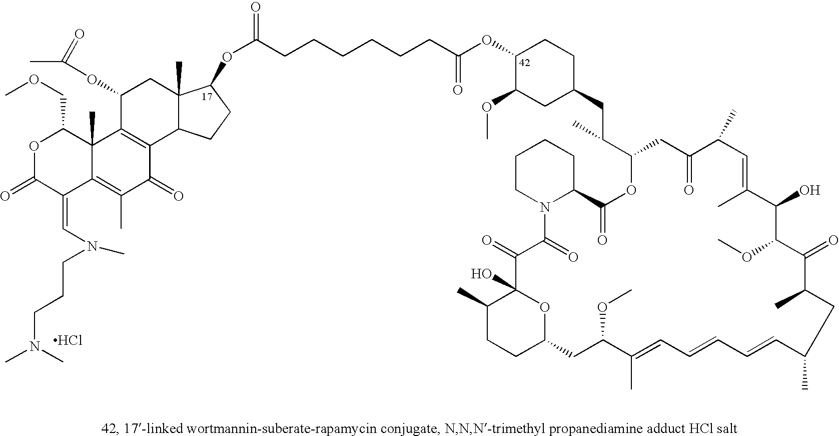

Other salts and adducts can be readily selected by one of skill in the art. The conjugates, as well as the rapamycin and wortmannin compounds may encompass tautomeric forms of the structures provided herein characterized by the bioactivity of the drawn structures.

The conjugates discussed herein also encompass “metabolites” which are unique products formed by processing the compounds by the cell or subject. Desirably, metabolites are formed in vivo.

In one embodiment, a salt and/or adduct of a free base conjugates described herein is desirable for improving the solubility, and thus, facilitating formulation of a conjugate. In another embodiment, improvement in the solubility of rapamycin is observed upon combination of a conjugate (e.g., a free base) with a buffer solution useful as a carrier for the conjugate. Such buffering solutions are described herein.

### Compositions and Uses of the Conjugates

In another aspect, the use of rapamycin-L-wortmannin conjugates in preparing a pharmaceutical composition is described. Typically, such a composition contains, at a minimum, the conjugate and a pharmaceutically acceptable carrier.

In one embodiment, a conjugate is mixed with a physiologically compatible liquid carrier for delivery through a desired route. The carrier can be a solvent or dispersion medium containing, for example, water, ethanol, polyol (e.g., glycerol, propylene glycol and liquid polyethylene glycol), suitable mixtures thereof, and vegetable oils. In one embodiment, the carrier may be readily selected from among buffered saline solution (e.g., phosphate buffered saline, Hepes buffered saline, Tris-buffered saline), many of which are commercially available.

The pharmaceutical compositions may contain one or more excipients. Excipients are added to the composition for a variety of purposes.

Diluents increase the bulk of a solid pharmaceutical composition, and may make a pharmaceutical dosage form containing the composition easier for the patient and caregiver to handle. Diluents for solid compositions include, for example, microcrystalline cellulose (e.g. Avicel® reagent), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit® reagent), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel® reagent), hydroxypropyl methyl cellulose (e.g. Methocel® reagent), liquid glucose, magnesium aluminum silicate, maltodextrin, methylcellulose, polymethacrylates, povidone (e.g. Kollidon® and Plasdone® reagents), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol® and Primellose® reagents), colloidal silicon dioxide, croscarmellose sodium, crospovidone (e.g. Kollidon® and Polyplasdone® reagents), guar gum, magnesium aluminum silicate, methyl cellulose, microcrystalline cellulose, polacrilin potassium, powdered cellulose, pregelatinized starch, sodium alginate, sodium starch glycolate (e.g. Explotab® reagent) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Solid and liquid compositions may also be dyed using any pharmaceutically acceptable colorant to improve their appearance and/or facilitate patient identification of the product and unit dosage level.

In liquid pharmaceutical compositions, the conjugate and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

A liquid composition may also contain a buffer such as gluconic acid, lactic acid, citric acid or acetic acid, sodium gluconate, sodium lactate, sodium citrate or sodium acetate. Selection of excipients and the amounts used may be readily determined by the formulation scientist based upon experience and consideration of standard procedures and reference works in the field.

The solid compositions include powders, granulates, aggregates and compacted compositions. The dosages include dosages suitable for oral, buccal, rectal, parenteral (including subcutaneous, intramuscular, and intravenous), inhalant and ophthalmic administration. The most suitable administration in any given case will depend on the nature and severity of the condition being treated. The dosages may be conveniently presented in unit dosage form and prepared by any of the methods well-known in the pharmaceutical arts.

Dosage forms include solid dosage forms such as tablets, powders, capsules, suppositories, sachets, troches and lozenges, as well as liquid syrups, suspensions and elixirs.

The dosage form may be a capsule containing the composition, for example, a powdered or granulated solid composition, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling may include any of the aforementioned blends and granulates that were described with reference to tableting, however, they are not subjected to a final tableting step.

### Uses and Products

In another aspect, an anti-neoplastic method is provided and comprises administering to a subject a pharmaceutically effective amount of a conjugate as described herein. Such a neoplasm is typically selected from prostate cancer, breast cancer, renal cancer, colon cancer, ovarian cancer, glioma, soft tissue sarcoma, neuroendocrine tumor of the lung, cervical cancer, uterine cancer, head and neck cancer, glioblastoma, non-small cell lung cancer, pancreatic cancer, lymphoma, melanoma, and small cell lung cancer.

In a combination therapy, the conjugate may be administered before, during, or after commencing therapy with another agent, as well as any combination thereof, i.e., before and during, before and after, during and after, or before, during and after commencing the other anti-cancer therapy.

When the anti-cancer therapy is radiation, the source of the radiation can be either external (external beam radiation therapy) or internal (brachytherapy) to the patient being treated. The dose of anti-cancer therapy administered to the patient depends on numerous factors, including, for example, the type of agent, the type and severity of the tumor being treated and the route of administration of the agent.

Optionally, the method provides for administering the conjugate in a combination regimen with another active component. Such active component may be readily selected by one of skill in the art from among, e.g., an immunomodulator (e.g., an immunostimulant or an immunosuppressant), an antineoplastic agent, or other desired component. When used in such a regimen, the conjugate may be administered prior to, simultaneously with, or following administration of the other active component. Further, the conjugate and the other active components may be delivered by the same route, or by different routes, of administration.

In one embodiment, the conjugate is administered in a regimen with an immunomodulator (e.g., an interferon, an interleukin (e.g., IL-2), or BCG). Suitable interferons are readily selected from among those known to those of skill in the art including, e.g., an interferon α, an interferon β, or an interferon γ. In one embodiment, the interferon is an interferon α. One interferon a (IFN α) is available commercially as the “Intron® A” reagent.

In another embodiment, the conjugate is administered in a regimen with an anti-VEGF monoclonal antibody. One suitable anti-VEGF monoclonal antibody is available, e.g., as AVASTIN.

As is typical with oncology treatments, dosage regimens are closely monitored by the treating physician, based on numerous factors including the severity of the disease, response to the disease, any treatment related toxicities, age, and health of the patient. Dosage regimens are expected to vary according to the route of administration.

Administration of the compositions may be oral, intravenous (i.v.), respiratory (e.g., nasal or intrabronchial), infusion, parenteral (besides i.v., such as intralesional, intraperitoneal and subcutaneous injections), intraperitoneal, transdermal (including all administration across the surface of the body and the inner linings of bodily passages including epithelial and mucosal tissues), and vaginal (including intrauterine administration). Other routes of administration are also feasible, such as via liposome-mediated delivery; topical, nasal, sublingual, uretheral, intrathecal, ocular or otic delivery, implants, rectally, intranasally.

It is projected that initial i.v. infusion dosages of the conjugate will be from about 5 to about 175 mg, or about 5 to about 25 mg, when administered on a weekly dosage regimen. It is projected that an oral dosage of a conjugate would be in the range of 10 mg/week to 250 mg/week, about 20 mg/week to about 150 mg/week, about 25 mg/week to about 100 mg/week, or about 30 mg/week to about 75 mg/week. For rapamycin, the projected oral dosage will be between 0.1 mg/day to 25 mg/day. Precise dosages will be determined by the administering physician based on experience with the individual subject to be treated.

In one embodiment, further included is a product or pharmaceutical pack containing one or more container(s) having one, one to four, or more unit(s) of a conjugate in unit dosage form and optionally, another active agent (e.g., an interferon or anti-VEGF monoclonal antibody). Such a product may contain other components, including, e.g., a diluent, carrier, syringe, and/or instructions for administration of the conjugate. Typically, pharmaceutical packs contain an anti-neoplastic dosage regimen for an individual mammal.

In another embodiment, pharmaceutical packs contain a course of anti-neoplastic treatment for one individual mammal comprising a container having a unit of a rapamycin—wortmannin conjugate in unit dosage form, and optionally, a container with another active agent. In other embodiments, the rapamycin is rapamycin, an ester (including a 42-ester), ether (including a 42-ether), tetrazole substituted (include 42-epi-tertazolyl), an amide, a carbonate, a carbamate of rapamycin. In another embodiment, the rapamycin is 42-O-(2-hydroxy)ethyl rapamycin. In another embodiment, the rapamycin is temsirolimus. In still another embodiment, the rapamycin is 42-epi-tetrazolyl rapamycin and the pack contains one or more container(s) comprising one, one to four, or more unit(s) of a temsirolimus (CCI-779)—wortmannin conjugate with the components described herein.

In some embodiments, the compositions are in packs in a form ready for administration. In other embodiments, the compositions are in concentrated form in packs, optionally with the diluent required to make a final solution for administration. In still other embodiments, the product contains a compound useful herein in solid form and, optionally, a separate container with a suitable solvent or carrier for the compound useful herein.

In still other embodiments, the above packs/kits include other components, e.g., instructions for dilution, mixing and/or administration of the product, other containers, syringes, needles, etc. Other such pack/kit components will be readily apparent to one of skill in the art.

### EXAMPLES

The following examples further illustrate the invention, but should not be construed to limit the scope of the invention in any way. It is to be understood and expected that variations in the principles of the invention herein disclosed may be made by one skilled in the art and it is intended that such modifications are to be included within the scope of the present invention.

### Example 1

### Synthesis of 42,17′-linked Rapamycin-Succinate-Wortmannin conjugate

### Method A:

To a solution of 17-hydroxywortmannin (430 mg, 1 mmol) in CH₂Cl₂ (10 mL) was added succinic anhydride (250 mg, 2.5 mmol), followed by DMAP (244 mg, 2 mmol). The mixture was then stirred at room temperature overnight. The crude material was purified by silica gel column eluting with hexane/acetone to give wortmannin 17-hemisuccinate (470 mg) as a white powder. MS (ESI) m/e 553 (M+Na).

A mixture of wortmannin 17-hemisuccinate (132.5 mg, 0.25 mmol), rapamycin 31-OTMS (259 mg, 0.26 mmol) and a catalytic amount of DMAP (5 mg) in MeCN (3 mL) was cooled to 0-5° C. and was treated with 1,3-Dicyclohexylcarbodiimide (62 mg, 0.3 mmol). The mixture was stirred at 0-5° C. for 16 hours. Aqueous sulfuric acid (0.5N, 1.5 ml,) was added dropwise and the mixture was stirred for 2 hours. EtOAc was added and the organic layer was separated. The organic layer was washed with water, 5% NaHCO₃ and brine. After the solvent was evaporated under vacuum, the crude residue was purified on a silica gel column to give the desired product as a white foam. MS (ESI) m/e 1426.

### Method B:

To a solution of 17-hydroxywortmannin (129 mg), 1,3-Dicyclohexylcarbodiimide (93 mg) and DMAP (5 mg) in CH₂Cl₂ at 0-5° C., was added rapamycin 42-hemisuccinate (304 mg). The mixture was stirred at 0-5° C. for 10 hours or until all starting material disappeared as monitored by TLC. Silica gel column purification of the reaction mixture furnished the desired product (342 mg) as a white foam.

### Example 2

### Synthesis of 42,17′-linked Rapamycin-Suberate-Wortmannin conjugate

### Method A:

To a solution of 17-hydroxywortmannin (430 mg, 1 mmol) in CH₂Cl₂ (10 mL) was added suberate anhydride (234 mg, 1.5 mmol), followed by DMAP (153 mg, 1.25 mmol). The mixture was then stirred at room temperature overnight. The crude was purified by silica gel column eluting with hexane/acetone to give wortmannin 17-hemisuberate (200 mg) as a white powder. MS (ESI) m/e 609 (M+Na).

A mixture of wortmannin 17-hemisuberate (147 mg, 0.25 mmol), rapamycin 31-OTMS (247 mg, 0.25 mmol) and catalytic amount of DMAP (6 mg) in MeCN (3 mL) was cooled to 0-5° C. and was treated with 1,3-Dicyclohexylcarbodiimide (72 mg, 0.35 mmol). The mixture was stirred at 0-5° C. for 16 hours. Aqueous sulfuric acid (0.5N, 1.5 mL) was added dropwise and the mixture was stirred for 2 hours. EtOAc was added and the organic layer was separated. The organic layer was washed with water, 5% NaHCO₃ and brine. After solvent was evaporated under vacuum. The crude was purified on a silica gel column to give desired product as white foam. MS (ESI) m/e 1482.

### Method B:

To a solution of 17-hydroxywortmannin (172 mg), 1,3-dicyclohexylcarbodiimide (124 mg) and DMAP (6 mg) in CH₂Cl₂ (6 mL) at 0-5° C., was added rapamycin 42-hemisuberate (428 mg). The mixture was stirred at 0-5° C. for 16 hours or until all starting material disappeared as monitored by TLC. Silica gel column purification of reaction mixture furnished desired product (370 mg) as white foam.

### Example 3

### Synthesis of 42,17′-linked Rapamycin-adipate-wortmannin conjugate

To a solution of 17-hydroxywortmannin (129 mg, 0.3 mmol), 1,3-Dicyclohexylcarbodiimide (93 mg, 0.45 mmol) and DMAP (5 mg) in CH₂Cl₂ (5 mL) at 0-5° C., was added rapamycin 42-hemiadipate (313 mg, 0.3 mmol). The mixture was stirred at 0-5° C. for 16 hours or until all starting material disappeared as monitored by TLC. Silica gel column purification of reaction mixture furnished desired product (230 mg) as white foam. MS (ESI) m/e 1454.

### Example 4

### Synthesis of amine adducts of 42,17′-linked Rapamycin-Wortmannin conjugates

### General Procedure:

To a 0° C. solution of rapamycin-wortmannin conjugates from examples 1-3 in organic solvent was added a solution of amine (0.11 mmol) in solvent. The mixture was stirred until the reaction was completed as monitored by TLC or HPLC. The solvent was removed in vacuo. The products were purified either by trituration with solvents or via chromatography on silica gel eluting with CH₂Cl₂—MeOH.

### Exemplary Example 4a

### Diallylamine adducts of 42,17′-linked Rapamycin-suberate-Wortmannin conjugates

A solution of 42,17′-linked rapamycin-suberate-wortmannin conjugate from example 2 (1.0 g) in TBME (30 mL) was cooled with an ice-bath and treated with diallylamine (0.15 mL). The mixture was stirred for 48 hours, concentrated to a volume of about 10 mL, and triturated with hexane (50 mL). The product was collected on a Buchner funnel as a yellow powder (985 mg). MS (ESI): (M⁻) 1580.

The representative compounds in Table 1 were synthesized by employing the appropriate amine and rapamycin-wortmannin conjugate.

TABLE 1

| | | | MS |
|---|---|---|---|
| | | | (ESI) |
| Amine | Conjugate | Conjugate Adduct Product | (M+) |
| diethylamine | 42,17′-linked | Diethylamine adduct of | 1528 |
| | rapamycin-adipate- | 42,17′-linked rapamycin- | |
| | wortmannin conjugate | adipate-wortmannin | |
| | | conjugate | |
| diallylamine | 42,17′-linked | Diallylamine adduct of | 1552 |
| | rapamycin-adipate- | 42,17′-linked rapamycin- | |
| | wortmannin conjugate | adipate-wortmannin | |
| | | conjugate | |

### Exemplary example 4b

### N,N,N′-Trimethyl-1,3-propanediamine adduct of 42,17′-linked rapamycin-suberate-wortmannin conjugate.

A solution of 42,17′-linked rapamycin-suberate-wortmannin conjugate from example 2 (7.70 g, 5.2 mmol) in TBME (225 mL) was cooled to −30 to −35° C. and treated with a solution of N,N,N′-trimethyl-1,3-propanediamine (694 mg, 5.98 mmol) in TBME (35 mL) over 45 minutes. The mixture was stirred for 1 hour at −30° C., then slowly warmed to −20° C. over 1 hour and stirred at −20° C. for another 1 hour. Hexane (280 mL) was then introduced while maintaining the temperature at −15 to −20° C. After stirring for 10 minutes, the precipitates were collected on a Buchner funnel and washed with cold Hexane/TBME (1:0.8), dried under vacuo, and the product was obtained as a yellow powder (7.8 g). MS (ESI): (M⁻) 1598.

The representative compounds in Table 2 were synthesized by employing N,N,N′-trimethyl-1,3-propanediamine and the appropriate rapamycin-wortmannin conjugate.

TABLE 2

| | | MS (ESI) |
|---|---|---|
| Conjugate | Conjugate Adduct Product | (M+) |
| 42,17′-linked | N,N,N′-trimethyl-1,3-propanediamine | 1543 |
| rapamycin-succinate- | adduct of 42,17′-linked rapamycin- | |
| wortmannin conjugate | succinate-wortmannin conjugate | |
| 42,17′-linked | N,N,N′-trimethyl-1,3-propanediamine | 1571 |
| rapamycin-adipate- | adduct of 42,17′-linked rapamycin- | |
| wortmannin conjugate | adipate-wortmannin conjugate | |

### Example 5

### Synthesis of 31,17′-linked rapamycin-suberate-wortmannin conjugate

A mixture of wortmannin 17-hemisuberate (293 mg, 0.5 mmol), rapamycin 42-OTBS (411 mg, 0.4 mmol) and a catalytic amount of DMAP (24.4 mg, 0.2 mmol) in 1,2-dichloroethane (4 mL) was cooled to 0-5° C. and was treated with 1,3-Diisopropylcarbodiimide (101 mg, 0.8 mmol). The mixture was stirred at 0-5° C. for 16 hours. Purification via silica gel gave 606 mg (95% yield) of a white powder.

This white powder (460 mg) was dissolved in MeCN (6 mL) and cooled with an ice-bath. 2N H₂SO₄ (2.5 mL) was added dropwise. After addition, the mixture was stirred for 4 hours at 0-5° C. EtOAc was added and the organic layer was separated. The organic layer was washed with water, 5% NaHCO₃ and brine, followed by evaporation of the solvent under vacuum. The crude product was purified on a silica gel column to give the desired product as white foam. MS (ESI): (M+Na)⁺1505.

### Example 6

### Synthesis of N,N,N′-trimethyl 1,3-propanediamine adduct of 31,17′-linked rapamycin-suberate-wortmannin

A solution of rapamycin-wortmannin conjugate from example 5 (70 mg) in TBME (0.4 mL) was cooled to −30° C. and treated with a solution of N,N,N′-trimethyl-1,3-propanediamine (7 mg) in TBME (0.1 mL). The mixture was stirred for 1 hour at −30° C. Hexane (0.5 mL) was added. After stirring for 10 minutes, the product was collected on a Buchner funnel as a yellow powder (70 mg). MS (ESI): (M⁺) 1600.

### Example 7

### Synthesis of diallyl amine adduct of 31,17′-linked rapamycin-suberate-wortmannin conjugate

A solution of rapamycin-wortmannin conjugate from example 5 (30 mg) in TBME (0.2 mL) was cooled to −20° C. and treated with a solution of diallylamine (3 mg) in TBME (0.1 mL). The mixture was stirred for 30 minutes at −20° C. The solvent was removed by a N₂ stream and triturated with hexane (1 mL). The product was collected on a Buchner funnel as a yellow powder (30 mg). MS (ESI): (M⁺+Na) 1602.

### Example 8

### Synthesis of 42,11′-linked rapamycin-suberate-wortmannin conjugate

A mixture of wortmannin 11-hemisuberate (271 mg, 0.5 mmol), rapamycin 31-OTMS (395 mg, 0.4 mmol) and a catalytic amount of DMAP (24.4 mg, 0.2 mmol) in 1,2-dichloroethane (4 mL) was cooled to 0-5° C. and treated with 1,3-Diisopropylcarbodiimide (101 mg, 0.8 mmol). The mixture was stirred at 0-5° C. for 5 hours, warmed to RT and stirred for another 12 hours. Purification via silica gel gave 340 mg (56% yield) of a white powder.

This white powder (340 mg) was dissolved in MeCN (5 mL), cooled with an ice-bath, and 0.5N H₂SO₄ (4 mL) was added dropwise. After addition, the mixture was stirred for 3 hours at 0-5° C. EtOAc was added and the organic layer was separated. The organic layer was washed with water, 5% NaHCO₃ and brine, followed by evaporation of the solvent under vacuum. The crude was purified on a silica gel column to give desired product as white foam (265 mg). MS (ESI): (M+Na)⁺1461.

### Example 9

### Synthesis of piperidine adduct of 42,11′-linked rapamycin-suberate-wortmannin

A solution of rapamycin-wortmannin conjugate from example 8 (30 mg) in CH₂Cl₂ (0.2 mL) was cooled with an ice-bath and treated with piperidine (4 mg). The mixture was stirred for 30 minutes. The solvent was removed by a N₂ stream and triturated with hexane (1 mL). The product was collected on a Buchner funnel as a yellow powder (30 mg). MS (ESI): (M⁺+Na) 1546.

### Example 10

### Synthesis of N,N-dimethyl-N′-ethyl-ethylenediamine adduct of 42,11′-linked rapamycin-suberate-wortmannin

A solution of rapamycin-wortmannin conjugate from example 8 (76 mg) in TBME (0.4 mL) was cooled to −30° C. and treated with a solution of N,N-dimethyl-N′-ethyl-ethylenediamine (8 mg) in TBME (0.1 mL). The mixture was stirred for 1 hour at −30° C. Hexane (1 mL) was added. After stirring for 10 minutes, the product was collected on a Buchner funnel as a yellow powder (80 mg). MS (ESI): (M⁺) 1556.

### Example 11

### Synthesis of 31,11′-linked rapamycin-suberate-wortmannin conjugate

A mixture of wortmannin 11-hemisuberate (271 mg, 0.5 mmol), rapamycin 42-OTBS (411 mg, 0.4 mmol) and a catalytic amount of DMAP (24.4 mg, 0.2 mmol) in 1,2-dichloroethane (4 mL) was cooled to 0-5° C. and treated with 1,3-Diisopropylcarbodiimide (101 mg, 0.8 mmol). The mixture was stirred at 0-5° C. for 4 hours, warmed to RT and stirred for another 12 hours. Purification via silica gel afforded 590 mg (95% yield) of a white powder.

This white powder (550 mg) was dissolved in MeCN (8 mL) and cooled with an ice-bath. 2N H₂SO₄ (3.5 mL) was added dropwise. After addition, the mixture was stirred for 4 hours at 0-5° C. EtOAc was added and the organic layer was separated. The organic layer was washed with water, 5% NaHCO₃ and brine, followed by evaporation of the solvent under vacuum. The crude product was purified on a silica gel column to give desired product as white foam (440 mg, 86%). MS (ESI): (M+Na)⁺1461.

### Example 12

### Synthesis of diethylamine adduct of 31,11′-linked rapamycin-suberate-wortmannin

A solution of rapamycin-wortmannin conjugate from example 11 (30 mg) in CH₂Cl₂ (0.2 mL) was cooled with an ice-bath and treated with diethylamine (2.2 mg). The mixture was stirred for 30 minutes. The solvent was removed by a N₂ stream and triturated with hexane (1 mL). The product was collected on a Buchner funnel as a yellow powder (30 mg). MS (ESI): (M⁺+Na) 1535.

### Example 13

### Synthesis of N,N,N′-trimethyl 1,3-propanediamine adduct of 31,11′-linked rapamycin-suberate-wortmannin

A solution of rapamycin-wortmannin conjugate from example 11 (72 mg) in TBME (0.4 mL) was cooled to −30° C. and treated with a solution of N,N,N′-trimethyl-1,3-propanediamine (8 mg) in TBME (0.1 mL). The mixture was stirred for 1 hour at −30° C. Hexane (1 mL) was added. After stirring for 10 minutes, the product was collected on a Buchner funnel as a yellow powder (72 mg). MS (ESI): (M⁺) 1555.

### Example 14

### In Vitro Cell Culture Growth Assays

Human tumor cell lines (Table 3) include prostate lines LNCap and PC3MM2, breast lines MDA468, MCF7, renal line HTB44 (A498), colon line HCT116, and ovarian line OVCAR3. Cells were plated in 96-well culture plates.

One day following plating, the following conjugates (inhibitors) were added to cells:
Compound A 42,17′-linked wortmannin-adipate-rapamycin conjugate, diethylamine adduct
Compound B 42,17′-linked wortmannin-adipate-rapamycin conjugate.
Compound C 42,17′-linked wortmannin-succinate-rapamycin conjugate, N,N,N′-trimethyl 1,3-propanediamine adduct
Compound D 42,17′-linked wortmannin-adipate-rapamycin conjugate, diallylamine adduct
Compound E 42,17′-linked wortmannin-adipate-rapamycin conjugate, N,N,N′-trimethyl 1,3-propanediamine adduct
Compound F 42,17′-linked wortmannin-suberate-rapamycin conjugate, N,N,N′-trimethyl 1,3-propanediamine adduct.

Three days after drug treatment, viable cell densities were determined by metabolic conversion (by viable cells) of the MTS dye (3-(4,5-dimethylthiazol-2-yl)-5-(3 carboxymethoxyphenyl)-2-(4-sulfophenyl)-2H-tetrazolium, inner salt), a well established cell proliferation assay. The assays were performed using an assay kit purchased from Promega Corp. (Madison, Wis.) following the protocol supplied with the kit. The MTS assay results were read in a 96-well plate reader by measuring absorbance at 490 nm. The effect of each treatment was calculated as percent of control growth relative to the vehicle-treated cells grown in the same culture plate. The drug concentration that conferred 50% inhibition of growth was determined as IC₅₀ (μg/mL). See, Table 3.

TABLE 3
IC₅₀ values (μg/mL) in inhibition of tumor cell growth in cell culture¹

| Compound | LNCap | PC2MM2 | MDA468 | MCF7 | HTB44 | HCT116 | OVCAR3 |
|---|---|---|---|---|---|---|---|
| A | 0.04 | 2.55 | 0.38 | 0.29 | 0.57 | >30 | 8.50 |
| B | 0.80 | 1.10 | 2.05 | 1.00 | 3.50 | 16.50 | 4.70 |
| C | 0.20 | 0.50 | 1.20 | 0.38 | 2.00 | 4.20 | 1.13 |
| D | 0.21 | 0.43 | 1.15 | 0.66 | 4.10 | 5.00 | 1.75 |
| E | 0.25 | 1.95 | 1.35 | 1.00 | 5.00 | 7.50 | 3.15 |
| F | 0.06 | 1.00 | 0.50 | 0.24 | 2.10 | 12.75 | 6.48 |

¹IC₅₀ values present the dose required for 50% reduction of cell growth for each of the indicated cancer types relative to vehicle treatment

### Example 15

### Female Mouse Whole Blood Stability Study

This study was conducted to determine the in vitro stability of 42,17′-linked wortmannin-suberate-rapamycin N,N,N′-trimethyl propanediamine (compound F in Example 14) in fresh female mouse blood collected in NaF/EDTA (ethylenediaminetetraacetic acid) tubes. A stock solution of the compound was prepared in the blood at a final concentration of 1000 ng/mL. The conjugate-spiked blood was incubated at 37° C. in a shaking water bath and aliquots were taken at 0, 5, 15, 30, 60, 90 and 120 minutes post-incubation.

A part of the sample was centrifuged to obtain plasma. Blood (100 μL) and plasma (100 μL) samples were extracted with acetonitrile (400 μL), vortexed for 2 minutes and centrifuged at 3400 rpm for 10 minutes. Supernatant (20 μL) was injected into an LC/MS/MS (Sciex API 4000™ instrument) for analysis of the conjugate and its hydrolyzed products. The HPLC system consisted of a FluoroSep-RPm Phenyl™ HS column. A gradient mobile phase of 0.1% formic acid in acetonitrile at a flow rate of 1 mL/min was used.

FIG. 1 shows the routes for the in vitro cleavage of 42,17′-linked-wortmannin-suberate-rapamycin conjugate, N,N,N′-trimethyl 1,3-propanediamine adduct in plasma.

### Example 16

### Xenograft Tumor Efficacy Study Methods

Female nude mice at 10 weeks of age were inoculated subcutaneously on the flank with 200 μL U87MG (human gliobastoma) tumor cell suspension. U87MG cells were suspended in full growth media and were implanted at 10 million cells per mouse. Mice were staged when tumors reached approximately 200 mm³ in sizes. Tumor bearing mice at staging (day 0) were randomized into treatment groups (n=10). The conjugates were formulated in D5W vehicle (glucose-water) and were dosed IV on day 0 and day 7.

The growth of tumors was monitored twice a week for the duration of the experiment. Tumor size was measured using sliding vernier calipers, and the tumor mass was calculated using the formula (length×Width²)/2.

Table 4 summarizes in vivo anticancer activity in U87MG glioma model of different conjugates. The experimental methods for drug preparation, dosing route, regimen, etc (listed in the Table 4) were similar to the experiments presented in the Figures. The data in Table 4 and the Figures were derived from multiple and completely different experiments. Within Table 4, the data contained separate experiments.

TABLE 4
In Vivo Anticancer Efficacy in U87MG Glioma Xenografts in Nude Mice

| | | | Tumor Volume (mm³) | | | | | |
|---|---|---|---|---|---|---|---|---|
| | Dose | | | | | | | S/T² |
| Group | (mg/kg) | | Day 0 | Day 3 | Day 7 | Day 10 | Day 14 | (D14) |
| Vehicle | n/a | mean³ | 185.3 | 274.5 | 498.8 | 1052.8 | 2220.9 | 9/10 |
| (D5W) | | se⁴ | 14.5 | 18.8 | 49.8 | 154.1 | 383.4 | |
| Compound C | 3 | mean | 193.0 | 209.6 | 437.2 | 691.8 | 1091.7 | 10/10 |
| | | se | 20.8 | 13.9 | 54.3 | 48.1 | 123.4 | |
| | | t/c⁵ | 1.04 | 0.76 | 0.88 | 0.66 | 0.49 | |
| | | p | 0.38429 | 0.00784 | 0.21948 | 0.02482 | 0.00853 | |
| | | value⁶ | | | | | | |
| Compound C | 15 | mean | 183.6 | 193.5 | 246.7 | 294.2 | | 10/10 |
| | | se | 15.8 | 21.0 | 30.3 | 37.6 | 34.1 | |
| | | t/c | 0.99 | 0.70 | 0.49 | 0.28 | 0.11 | |
| | | p | 0.46884 | 0.00501 | 0.00026 | 0.00009 | 0.00004 | |
| | | value | | | | | | |
| Compound D | 3 | mean | 185.1 | 244.5 | 435.4 | 636.4 | 1050.1 | 10/10 |
| | | se | 19.5 | 23.4 | 30.9 | 51.7 | 166.3 | |
| | | t/c | 1.00 | 0.89 | 0.87 | 0.60 | 0.47 | |
| | | p | | 0.16503 | 0.15030 | 0.01022 | 0.00635 | |
| | | value | | | | | | |
| Compound D | 15 | mean | 181.5 | 186.0 | | 242.5 | 254.6 | 10/10 |
| | | se | 12.7 | 18.4 | 23.9 | 42.8 | 45.0 | |
| | | t/c | 0.98 | 0.68 | | 0.23 | 0.11 | |
| | | p | 0.42313 | 0.00174 | 0.00004 | 0.00005 | 0.00004 | |
| | | value | | | | | | |
| Compound E | 3 | mean | 182.7 | 214.7 | 340.2 | 471.6 | 595.9 | 10/10 |
| | | se | 17.8 | 25.7 | 32.4 | 53.2 | 57.7 | |
| | | t/c | 0.99 | 0.78 | 0.68 | 0.45 | 0.27 | |
| | | p | 0.45557 | 0.03838 | 0.00874 | 0.00121 | 0.00030 | |
| | | value | | | | | | |
| Compound E | 15 | mean | 187.5 | 168.0 | 181.6 | 165.8 | 162.8 | 10/10 |
| | | se | 13.4 | 12.2 | 15.7 | 15.5 | 17.7 | |
| | | t/c | 1.01 | 0.61 | 0.36 | 0.16 | 0.07 | |
| | | p | 0.45633 | 0.00008 | 0.00001 | 0.00001 | 0.00002 | |
| | | value | | | | | | |

²Survival over total in group
³Mean tumor mass of the group
⁴Standard error
⁵Treated over control
⁶A p value less than 0.05 indicates a statistically significant inhibition of tumor growth

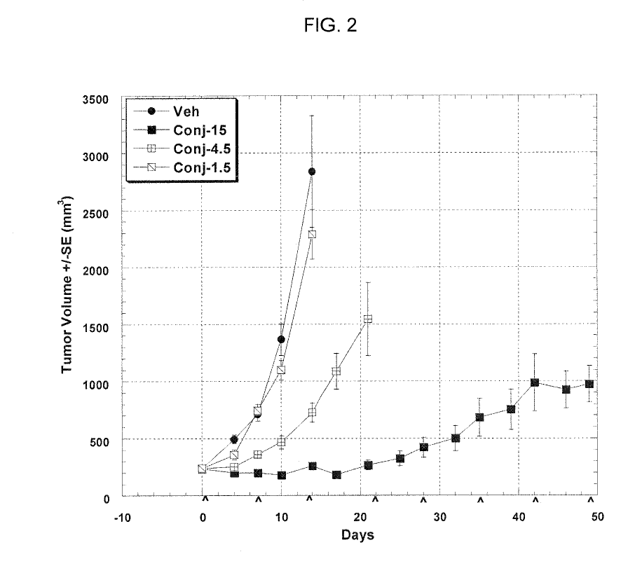

FIG. 2 shows the antitumor activity for 42,17′-linked wortmannin-succinate-rapamycin N,N,N′-trimethyl 1,3-propanediamine adduct following i.v. dosing 1× weekly for 2 rounds at 1.5 mg/kg ( [symbol] ), and 4.5 mg/kg ( [symbol] ), or 8 rounds at 15 mg/kg (▪), with vehicle () serving as negative control. This data shows improved inhibition of tumor cell growth at the lowest dose for two rounds, with some improvement at the 4.5 mg/kg for 2 weeks. Significant improvement was observed at the highest dose over 8 rounds. This data shows the sustained efficacy of 42,17′-linked wortmannin-adipate-rapa conjugate, N,N,N′-trimethyl 1,3-propanediamine adduct against U87MG glioma xenograph in multi-cycle treatment.

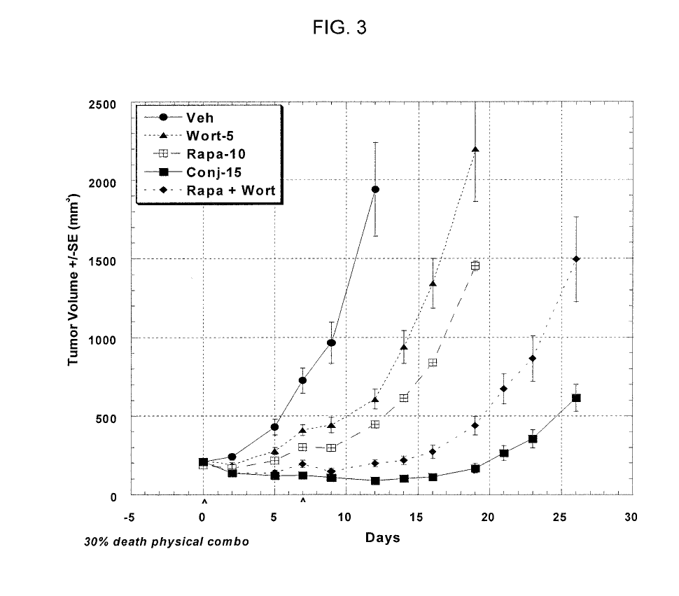

FIG. 3 shows the antitumor efficacy of various conjugates in comparison to ( [symbol] ) rapamycin alone (10 mg/kg), (solid triangle, ▴) 17-hydroxywortmannin N, N,N′-trimethyl 1,3-propanediamine adduct alone (5 mg/kg), (▪), 42,17-linked wortmannin-suberate-rapamycin conjugate, N,N,N′-trimethyl 1,3-propanediamine adduct (15 mg/kg), and (solid diamond) a physical combination of rapamycin (10 mg/kg) and 17-hydroxywortmannin N,N,N′-trimethyl 1,3-propanediamine adduct (5 mg/kg), when dosed 1× weekly for 2 rounds.

These data show that the conjugate is at least as active as the physical combination. However, the physical combination of rapamycin and the wortmannin were poorly tolerated, resulting in a 30% death rate.

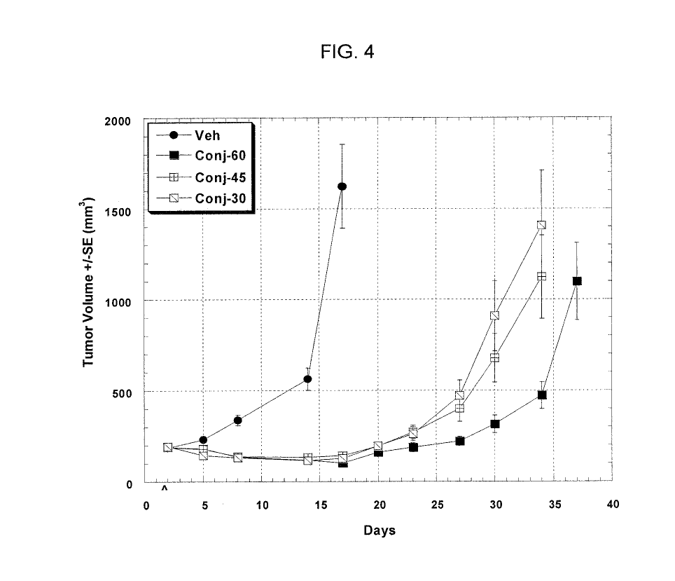

FIG. 4 shows the efficacy of 42,17′-linked wortmannin-suberate-rapamycin conjugate, N,N,N′-trimethyl 1,3-propanediamine adduct, against U87MG Glioma xenograph after a single dose at three dosing concentrations (30 mg/kg ( [symbol] ), 45 mg/kg ( [symbol] ), and 60 mg/kg (▪), as compared to vehicle (). In addition to the weekly dosing regimen, the data in FIG. 4 demonstrate that the conjugate is also effective in the intermittent regimen (e.g., 1× every 2 weeks, 1× monthly, etc).

### Example 17

### In Vivo Anticancer Efficacy in HT29 Colon Tumor Model

The effect of 42,17′-linked wortmannin-suberate-rapamycin conjugate, N,N,N′-trimethyl 1,3-propanediamine adduct, prepared as described in Example 4 was studied in a model of human colon cancer.

The 42,17′-linked wortmannin-suberate-rapamycin conjugate, N,N,N′-trimethyl 1,3-propanediamine adduct (▪, 15 mg/kg), rapamycin (▴, 10 mg/kg), 17-hydroxywortmannin N,N,N′-trimethyl 1,3-propanediamine adduct (▾, 5 mg/kg) and a physical combination of rapamycin (10 mg/kg) and 17-hydroxywortmannin N,N,N′-trimethyl 1,3-propanediamine adduct (5 mg/kg) were dosed 1× weekly for four rounds against a HT29 colon tumor xenograph. Vehicle served as a negative control ().

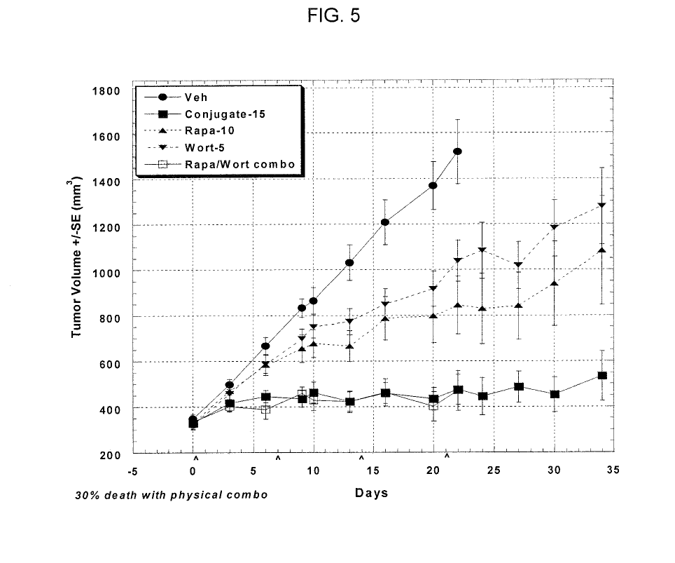

As can be seen in FIG. 5, while early results showed significant tumor reduction with the physical combination of rapamycin and the 17-hydroxywortmannin ( [symbol] ), a 30% death rate was observed with this combination. In contrast, the conjugate was better tolerated (▪), and significant tumor reduction in the group receiving the conjugate was observed for the duration of the study.

### Example 18

### In Vivo Anticancer Efficacy in HTB44/A498 Renal Cell Carcinoma Model

This study utilized a model of renal cancer performed as previously described. See, e.g., Yu K, et al., PWT-458, A Novel Pegylated-17-Hydroxywortmannin, Inhibits Phosphatidylinositol 3-Kinase Signaling and Suppresses Growth of Solid Tumors. Cancer Biol Ther. May 28, 2005; 4(5).

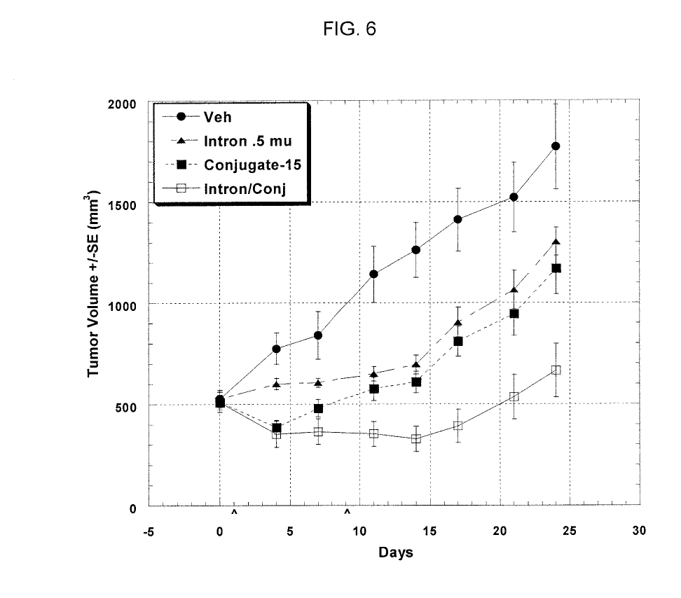

FIG. 6 shows the efficacy of 42,17′-linked wortmannin-suberate-rapamycin conjugate, N,N,N′-trimethyl 1,3-propanediamine adduct (▪) dosed i.v. 1× weekly at 15 mg/kg, 2 rounds), (▴) Intron® A reagent (doses ip 3× weekly, 0.5 mU, 2 weeks) and ( [symbol] ) a physical combination of 42,17-linked wortmannin-suberate-rapamycin conjugate, N,N,N′-trimethyl 1,3-propanediamine adduct (15 mg/kg) and Intron® A reagent (0.5 mU) against A498 renal cell carcinoma xenograph. The conjugate alone showed increased antitumor activity as compared to vehicle and Intron® A reagent alone. Significant antitumor activity was observed for the combination of Introng A reagent and conjugate.

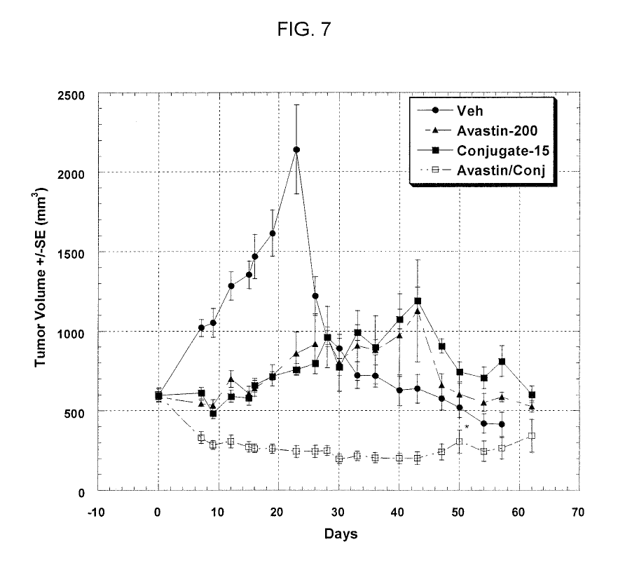

FIG. 7 shows the efficacy against A498 renal cell carcinoma xenograph of 42,17-linked wortmannin-suberate-rapamycin conjugate, N,N,N′-trimethyl 1,3-propanediamine adduct (▪, 15 mg/kg), the Avasting drug (▴, 200 μg), and a combination ( [symbol] ) of 42,17′-linked wortmannin-suberate-rapamycin conjugate, N,N,N′-trimethyl 1,3-propanediamine adduct and the Avastin® drug, which were dosed intravenously 1× weekly for six rounds. On day 23 the Vehicle group () was redosed with a combination 42,17′-linked wortmannin-suberate-rapamycin N,N,N′-trimethyl 1,3-propanediamine adduct (30 mg/kg) and the Avastin® drug (200 μg) for five rounds. On day 43, the 42,17′-linked wortmannin-suberate-rapamycin conjugate, N,N,N′-trimethyl 1,3-propanediamine adduct group (30 mg/kg) and the Avastin® drug group (200 μg) were redosed with a combination of 42,17′-linked wortmannin-suberate-rapamycin conjugate, N,N,N′-trimethyl 1,3-propanediamine adduct and the Avasting drug for three rounds.

The data in FIG. 7 suggest that the conjugate, when combined with the Avastin® drug, can cause significant regression of very large size tumors. Regression at this level has not been previously observed.

All publications cited in this specification, and the sequence listing, are incorporated herein by reference. While the invention has been described with reference to particular embodiments, it will be appreciated that modifications can be made without departing from the spirit of the invention. Such modifications are intended to fall within the scope of the appended claims.

1. A conjugate having the formula:
Rap-L-Wort
or a pharmaceutically acceptable salt or hydrate thereof,
wherein Rap is a rapamycin; Wort is a wortmannin, and L is a linker which is bound to the rapamycin and the wortmannin.

2. The conjugate according to claim 1, wherein L is removed in whole or part in vivo from one or both of Rap or Wort.

3. The conjugate according to claim 2, wherein L is hydrolysable or enzymatically cleaved.

4. The conjugate according to claim 1, wherein L is of formula (V):
-Z¹-X-Z²- (V)
wherein:
Z¹ and Z² are independently selected from the group consisting of —O—, —N(R⁰)—, —S—, —OC(═O)—, —OC(═O)O—, —N(R⁰)C(═O)—, —OC(═O)N(R⁰)—, —N(R⁰)C(═O)N(R⁰)—, —OC(═S)N(R⁰)—, —N(R⁰)C(═S)N(R⁰)—, ═N—N(R⁰)—, and a bond;
R⁰ at each occurrence is independently selected from the group consisting of H, alkyl, alkenyl, and aryl; and
X is selected from the group consisting of cycloalkyl, aryl, alkylarylalkyl, heteroaryl, a heterocyclic group, a hydrocarbon chain having from 1 to 16 carbon atoms which may be branched, unbranched, saturated or unsaturated, may be optionally substituted with one or more of oxy, amine, sulfide, alkyl, alkenyl, aryl, alkoxy, hydroxyl, and halogen, and may be optionally interrupted by one or more ether (—O—), amine (—NH—), sulfide (—S—), —S(O)ₙ—, —N(R⁰)—, —C(═O)N(R⁰)—, or —OC(═O)N(R⁰)— and n is 0 to 2 or combinations thereof.

5. The conjugate according to claim 4, wherein when Z¹ or Z² is selected from the group consisting of —O—, —N(R⁰)—, —S—, —OC(═O)—, —OC(═O)O—, —N(R⁰)C(═O)—, —OC(═O)N(R⁰)—, —N(R⁰)C(═O)N(R⁰)—, —OC(═S)N(R⁰)—, —N(R⁰)C(═S)N(R⁰)—, and ═N—N(R⁰)—, wherein the group through which L is bound to the Rap or wort does not provide a further O group.

6. The conjugate according to claim 4, wherein X is selected from the group consisting of an alkyl chain of 1 to 16 carbon atoms interrupted by one or more groups selected from the group consisting of O, —S(O)ₙ—, —N(R⁰)—, —OC(═O)—, —OC(═O)O—, —C(═O)N(R⁰)—, and —OC(═O)N(R⁰)—, and n is 0 to 2.

7. The conjugate according to claim 4, wherein the linker has the formula:

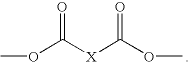

8. The conjugate according to claim 7, wherein X is a hydrocarbon chain of the formula —(CH₂)ₙ—, where n is 1-16.

9. The conjugate according to claim 4, wherein Z¹ and Z² are a bond and X is an alkyl chain of 1 to 10 carbon atoms or an alkyl chain of 1 to 10 carbon atoms substituted with one, two, or more oxy groups.

10. The conjugate according to claim 4, wherein X is selected from the group consisting of C₁-C₈ alkyl, C₂-C₈ alkenyl, (CH₂CH₂O)ₙ, CH₂OCH₂, cycloalkyl, aryl, and a heterocyclic group.

11. The conjugate according to claim 7, wherein X is selected from the group consisting of (CH₂)₂, (CH₂)₃, (CH₂)₄, and (CH₂)₆.

12. The conjugate according to claim 1, wherein the rapamycin has the core structure of formula (IIa): 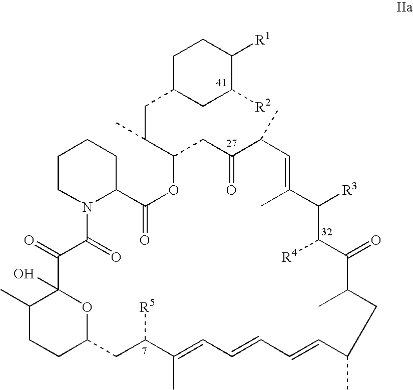
wherein:
R¹ is selected from the group consisting of OH, an ester, an ether, an amide, a carbonate, a carbamate, a phosphate, a tetrazole, and a point of attachment to L;
R² is selected from the group consisting of OH, an ester, an ether, and a point of attachment to L;
R³ is selected from the group consisting of OH, an ester, an ether, an amide, a carbonate, a carbamate, and a point of attachment to L;
R⁴ is selected from the group consisting of H, OH, an ester, an ether, and a point of attachment to L;
R⁵ is selected from the group consisting of OH, an ester, an ether, and a point of attachment to L;
wherein at least one of R¹, R², R³, R⁴, and R⁵ is the point of attachment to L,
or pharmaceutically acceptable salts of structure IIa; provided that the rapamycin is not 41-desmethoxyrapamycin.

13. The conjugate according to claim 1, wherein Rap is rapamycin or a rapamycin 42-ester.

14. The conjugate according to claim 13, wherein the rapamycin is a rapamycin 42-ester with 3-hydroxy-2-(hydroxymethyl)-2-methylpropionic acid.

15. The conjugate according to claim 1, wherein the wortmannin has the core structure of formula (Ia1): 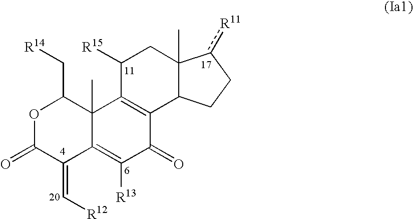
wherein:
R¹¹ is selected from the group consisting of O, OH, an ester, a carbonate, a carbamate, an ether, and a point of attachment to L;
R¹² and R¹³ are bound together via an O heteroatom, or
R¹² is selected from the group consisting of an ester, an ether, a thioether, a thioester, and a point of attachment to L;
R¹³ is selected from the group consisting of OH, an ester, a carbonate, a carbamate, an ether, and a point of attachment to L;
R¹⁴ is selected from the group consisting of OH, an ester, an ether, and a point of attachment to L;
R¹⁵ is selected from the group consisting of O, OH, an ester, a carbonate, a carbamate, and a point of attachment to L;
wherein at least one of R¹¹, R¹², R¹³, R¹⁴, and R¹⁵ is the point of attachment to L.

16. The conjugate according to claim 15, wherein:
R¹² is NR^{a}R^{b};
R^{a} and R^{b} are independently selected from the group consisting of H, alkyl, alkenyl, alkynyl, -(alkyl)-O-(alkyl)-, -(alkyl)-NR^{c}R^{d}—, -(alkyl)-C(═O)NR^{c}R^{d}—, cycloalkyl, aryl, and a heterocyclic group, with the proviso that both R^{a} and R^{b} cannot be H;
or R^{a} and R^{b} may be taken together to form a three to seven membered heterocyclic ring having up to 3 heteroatoms which is optionally substituted by from 1 to 3 substituents independently selected from the group consisting of halogen, hydroxyl, thio, alkyl, alkenyl, alkoxy, oxo, amino, cyano, C₁-C₃ perfluoroalkyl, alkylaryl, alkylheteroaryl, aryl, and heteroaryl;
R^{c} and R^{d} are independently selected from the group consisting H, alkyl, alkenyl, alkynyl, aryl, cycloalkyl, and heterocycyl; or
R^{c} and R^{d} are taken together to form a three to seven membered cyclic or heterocyclic ring having up to 3 heteroatoms which is optionally substituted by 1 to 3 substituents independently selected from the group consisting of halogen, hydroxyl, thio, alkyl, alkenyl, alkoxy, oxo, amino, cyano and C₁-C₃ perfluoroalkyl.

17. The conjugate according to claim 16, wherein:
R^{a} is H and R^{b} is phenyl; or
R^{a} and R^{b} are a lower alkyl.

18. The conjugate according to claim 16, wherein R¹² is selected from the group consisting of diethylamine, diallylamine, N,N,N′-trimethyl-1,3-propanediamine, piperidine, and N,N-dimethyl-N′-ethyl-ethylenediamine and R¹³ is —OH.

19. The conjugate according to claim 1, wherein the conjugate is selected from the group consisting of:
wortmannin-glutarate-rapamycin conjugate;
wortmannin-suberate-rapamycin conjugate;
wortmannin-diglycolinate-rapamycin conjugate;
wortmannin-adipate-rapamycin conjugate;
wortmannin-succinate-rapamycin conjugate;
wortmannin-suberate-rapamycin conjugate, N,N,N′-trimethyl 1,3-propanediamine adduct;
wortmannin-adipate-rapamycin conjugate, diethylamine adduct;
wortmannin-succinate-rapamycin conjugate, N,N,N′-trimethyl 1,3-propanediamine adduct;
wortmannin-adipate-rapamycin conjugate, diallylamine adduct;
wortmannin-adipate-rapamycin conjugate, N,N,N′-trimethyl 1,3-propanediamine adduct;
wortmannin-suberate-rapamycin conjugate, piperidine adduct;
wortmannin-suberate-rapamycin conjugate, N,N-dimethyl-N′-ethyl-ethylenediamine adduct;
wortmannin-suberate-rapamycin conjugate, diallylamine adduct;
wortmannin-suberate-rapamycin conjugate, diethylamine adduct;
and pharmaceutically acceptable salts thereof.

20. A pharmaceutical composition comprising a conjugate according to claim 1 and a pharmaceutically acceptable carrier.

21. A method of treating a neoplasm comprising administering to a subject a pharmaceutically effective amount of a conjugate according to claim 1.

22. The method according to claim 21, wherein the neoplasm is selected from the group consisting of prostate cancer, breast cancer, renal cancer, colon cancer, ovarian cancer, glioma, soft tissue sarcoma, neuroendocrine tumor of the lung, cervical cancer, uterine cancer, head and neck cancer, glioblastoma, non-small cell lung cancer, pancreatic cancer, lymphoma, melanoma, and small cell lung cancer.

23. The method according to claim 21, wherein said method further comprises administering the conjugate in a combination regimen with an interferon or an anti-VEGF monoclonal antibody.

24. The method according to claim 23, wherein the interferon is an interferon α.

25. The method according to claim 23, wherein the conjugate is administered prior to, simultaneously with, or following administration of said interferon or said anti-VEGF monoclonal antibody.

26. The method according to claim 22, wherein the conjugate is administered directly to the neoplasm.
